(19) United States
(12) Patent Application Publication
(10) Pub. No.: US 2026/0183438 A1
(43) Pub. Date: Jul. 2, 2026

(54) FLUID STERILIZING APPARATUS

(71) Applicant: NICHIA CORPORATION, Tokushima (JP)

(72) Inventors: Yaser Abdelsamed, Granville, OH (US); Evan Cooke-DiBarto, Suwanee, GA (US); Scott Akin, Suwanee, GA (US)

(21) Appl. No.: 19/244,519

(22) Filed: Jun. 20, 2025

Related U.S. Application Data
(60) Provisional application No. 63/738,921, filed on Dec. 26, 2024.

(51) Int. Cl. A61L 2/10; A61L 2/24

(52) U.S. Cl. CPC A61L 2/10; A61L 2/24; A61L 2202/11

(57) ABSTRACT

A fluid sterilizing apparatus includes a first optoelectronic element, a first optoelectronic element for emitted light, and a first optoelectronic element for reflected light; an optoelectronic element for transmitted light disposed to face the first optoelectronic element, the first optoelectronic element for emitted light, and the first optoelectronic element for reflected light; a chamber including an inner space that allows passage of a fluid to be irradiated with ultraviolet light emitted from the first optoelectronic element; a first light-transmitting member; and a control unit including a processor configured to control emission of the ultraviolet light from the first optoelectronic element upon receiving a first reflected light reception signal, a first emitted light reception signal, and a first transmitted light reception signal.

### CROSS-REFERENCE TO RELATED APPLICATIONS

This application is based upon and claims priority to U.S. Provisional Ser. No. 63/738,921 , filed on Dec. 26, 2024, the entire contents of which are incorporated herein by reference.

### BACKGROUND

The present disclosure relates to a fluid sterilizing apparatus.

Fluid sterilizing apparatuses using an ultraviolet light emitting diode (ultraviolet LED) are known. For example, U.S. Pat. No. 11,473,972 discloses a fluid sterilizing apparatus including: a solid-state electromagnetic radiation source; a reaction chamber positioned such that a fluid in the reaction chamber is exposed to electromagnetic radiation of ultraviolet light radiated from the solid-state electromagnetic radiation source; and an electromagnetic radiation sensor, such as an optoelectronic element or the like, spaced apart from the solid-state electromagnetic radiation source and configured to sense any electromagnetic radiation emitted from the solid-state electromagnetic radiation source and reflected by at least one surface.

### SUMMARY

A fluid sterilizing apparatus according to an embodiment of the present disclosure includes: a first optoelectronic element, a first optoelectronic element for emitted light, and a first optoelectronic element for reflected light; an optoelectronic element for transmitted light disposed to face the first optoelectronic element, the first optoelectronic element for emitted light, and the first optoelectronic element for reflected light; a chamber including an inner space that allows passage of a fluid to be irradiated with ultraviolet light emitted from the first optoelectronic element; a first light-transmitting member; and a control unit including a processor configured to control emission of the ultraviolet light from the first optoelectronic element upon receiving a first reflected light reception signal, a first emitted light reception signal, and first transmitted light reception signal. The chamber is disposed between the first optoelectronic element and the optoelectronic element for transmitted light, the chamber is disposed between the first optoelectronic element for emitted light and the optoelectronic element for transmitted light, and the chamber is disposed between the first optoelectronic element for reflected light and the optoelectronic element for transmitted light. The first light-transmitting member is disposed between the first optoelectronic element and the inner space of the chamber, the first light-transmitting member is disposed between the first optoelectronic element for emitted light and the inner space of the chamber, and the first light-transmitting member is disposed between the first optoelectronic element for reflected light and the inner space of the chamber. The first reflected light reception signal is generated from the first optoelectronic element for reflected light upon receiving the ultraviolet light emitted from the first optoelectronic element and reflected by the first light-transmitting member. The first emitted light reception signal is generated from the first optoelectronic element for emitted light upon receiving a part of the ultraviolet light emitted from the first optoelectronic element. The first transmitted light reception signal is generated from the optoelectronic element for transmitted light upon receiving the ultraviolet light emitted from the first optoelectronic element and subsequently transmitted through the first light-transmitting member and the fluid.

A fluid sterilizing apparatus according to another embodiment of the present disclosure includes: one or more first optoelectronic elements, a first optoelectronic element for emitted light, and a first optoelectronic element for reflected light; one or more second optoelectronic elements, a second optoelectronic element for emitted light, and a second optoelectronic element for reflected light that are disposed to face the one or more first optoelectronic elements, the first optoelectronic element for emitted light, and the first optoelectronic element for reflected light; a chamber including an inner space that allows passage of a fluid to be irradiated with ultraviolet light emitted from the one or more first optoelectronic elements; a first light-transmitting member; a second light-transmitting member; and a control unit including a processor configured to alternately switch between a state I in which the one or more first optoelectronic elements emit the ultraviolet light and the first optoelectronic element for emitted light, the first optoelectronic element for reflected light, the one or more second optoelectronic elements, the second optoelectronic element for emitted light, and the second optoelectronic element for reflected light do not emit the ultraviolet light, and a state II in which the one or more second optoelectronic elements emit the ultraviolet light and the one or more first optoelectronic elements, the first optoelectronic element for emitted light, the first optoelectronic element for reflected light, the second optoelectronic element for emitted light, and the second optoelectronic element for reflected light do not emit the ultraviolet light. The chamber is disposed between (A) and (B), wherein (A) is a combination of the one or more first optoelectronic elements, the first optoelectronic element for emitted light, and the first optoelectronic element for reflected light, and (B) is a combination of the one or more second optoelectronic elements, the second optoelectronic element for emitted light, and the second optoelectronic element for reflected light. The first light-transmitting member is disposed between the one or more first optoelectronic elements and the inner space of the chamber, the first light-transmitting member is disposed between the first optoelectronic element for emitted light and the inner space of the chamber, and the first light-transmitting member is disposed between the first optoelectronic element for reflected light and the inner space of the chamber. The second light-transmitting member is disposed between the one or more second optoelectronic elements and the inner space of the chamber, the second light-transmitting member is disposed between the second optoelectronic element for emitted light and the inner space of the chamber, and the second light-transmitting member is disposed between the second optoelectronic element for reflected light and the inner space of the chamber. In the state I, the first optoelectronic element for reflected light outputs a first reflected light reception signal upon receiving the ultraviolet light emitted from the one or more first optoelectronic elements and reflected by the first light-transmitting member, the first optoelectronic element for emitted light outputs a first emitted light reception signal upon receiving a part of the ultraviolet light emitted from the one or more first optoelectronic elements, the second optoelectronic element for reflected light outputs a first transmitted light reception signal upon receiving the ultraviolet light emitted from the one or more first optoelectronic elements and subsequently transmitted through the first light-transmitting member, the fluid, and the second light-transmitting member, and the processor controls emission of the ultraviolet light from the one or more first optoelectronic elements based on the first reflected light reception signal, the first emitted light reception signal, and the first transmitted light reception signal. In the state II, the second optoelectronic element for reflected light outputs a second reflected light reception signal upon receiving the ultraviolet light emitted from the one or more second optoelectronic elements and reflected by the second light-transmitting member, the second optoelectronic element for emitted light outputs a second emitted light reception signal upon receiving a part of the ultraviolet light emitted from the one or more second optoelectronic elements, the first optoelectronic element for reflected light outputs a second transmitted light reception signal upon receiving the ultraviolet light emitted from the one or more second optoelectronic elements and subsequently transmitted through the second light-transmitting member, the fluid, and the first light-transmitting member, and the processor controls emission of the ultraviolet light from the one or more second optoelectronic elements based on the second reflected light reception signal, the second emitted light reception signal, and the second transmitted light reception signal.

A fluid sterilizing apparatus according to a yet another embodiment of the present disclosure includes: a first optoelectronic element, a third optoelectronic element, a first optoelectronic element for emitted light, and a third optoelectronic element for emitted light; a second optoelectronic element, a fourth optoelectronic element, a second optoelectronic element for emitted light, and a fourth optoelectronic element for emitted light that are disposed to face the first optoelectronic element, the third optoelectronic element, the first optoelectronic element for emitted light, and the third optoelectronic element for emitted light; a chamber including an inner space that allows passage of a fluid to be irradiated with ultraviolet light emitted from the first optoelectronic element; a first light-transmitting member; a second light-transmitting member; and a control unit including a processor configured to sequentially switch between a first state in which the first optoelectronic element emits the ultraviolet light, and the third optoelectronic element, the first optoelectronic element for emitted light, the third optoelectronic element for emitted light, the second optoelectronic element, the fourth optoelectronic element, the second optoelectronic element for emitted light, and the fourth optoelectronic element for emitted light do not emit the ultraviolet light, a second state in which the third optoelectronic element emits the ultraviolet light, and the first optoelectronic element, the first optoelectronic element for emitted light, the third optoelectronic element for emitted light, the second optoelectronic element, the fourth optoelectronic element, the second optoelectronic element for emitted light, and the fourth optoelectronic element for emitted light do not emit the ultraviolet light, a third state in which the second optoelectronic element emits the ultraviolet light, and the first optoelectronic element, the third optoelectronic element, the first optoelectronic element for emitted light, the third optoelectronic element for emitted light, the fourth optoelectronic element, the second optoelectronic element for emitted light, and the fourth optoelectronic element for emitted light do not emit the ultraviolet light, and a fourth state in which the fourth optoelectronic element emits the ultraviolet light, and the first optoelectronic element, the third optoelectronic element, the first optoelectronic element for emitted light, the third optoelectronic element for emitted light, the second optoelectronic element, the second optoelectronic element for emitted light, and the fourth optoelectronic element for emitted light do not emit the ultraviolet light. The chamber is disposed between (A) and (B), wherein (A) is a combination of the first optoelectronic element, the third optoelectronic element, the first optoelectronic element for emitted light, and the third optoelectronic element for emitted light, and (B) is a combination of the second optoelectronic element, the fourth optoelectronic element, the second optoelectronic element for emitted light, and the fourth optoelectronic element for emitted light. The first light-transmitting member is disposed between the first optoelectronic element and the inner space of the chamber, the first light-transmitting member is disposed between the third optoelectronic element and the inner space of the chamber, the first light-transmitting member is disposed between the first optoelectronic element for emitted light and the inner space of the chamber, and the first light-transmitting member is disposed between the third optoelectronic element for emitted light and the inner space of the chamber. The second light-transmitting member is disposed between the second optoelectronic element and the inner space of the chamber, the second light-transmitting member is disposed between the fourth optoelectronic element and the inner space of the chamber, the second light-transmitting member is disposed between the second optoelectronic element for emitted light and the inner space of the chamber, and the second light-transmitting member is disposed between the fourth optoelectronic element for emitted light and the inner space of the chamber. In the first state, the third optoelectronic element outputs a third reflected light reception signal upon receiving the ultraviolet light emitted from the first optoelectronic element and reflected by the first light-transmitting member, the first optoelectronic element for emitted light outputs a first emitted light reception signal upon receiving a part of the ultraviolet light emitted from the first optoelectronic element, the second optoelectronic element or the fourth optoelectronic element outputs a first transmitted light reception signal upon receiving the ultraviolet light emitted from the first optoelectronic element and subsequently transmitted through the first light-transmitting member, the fluid, and the second light-transmitting member, and the processor controls emission of the ultraviolet light from the first optoelectronic element based on the third reflected light reception signal, the first emitted light reception signal, and the first transmitted light reception signal. In the second state, the first optoelectronic element outputs a first reflected light reception signal upon receiving the ultraviolet light emitted from the third optoelectronic element and reflected by the first light-transmitting member, the third optoelectronic element for emitted light outputs a third emitted light reception signal upon receiving a part of the ultraviolet light emitted from the third optoelectronic element, the second optoelectronic element or the fourth optoelectronic element outputs a third transmitted light reception signal upon receiving the ultraviolet light emitted from the third optoelectronic element and subsequently transmitted through the first light-transmitting member, the fluid, and the second light-transmitting member, and the processor controls emission of the ultraviolet light from the third optoelectronic element based on the first reflected light reception signal, the third emitted light reception signal, and the third transmitted light reception signal. In the third state, the fourth optoelectronic element outputs a fourth reflected light reception signal upon receiving the ultraviolet light emitted from the second optoelectronic element and reflected by the second light-transmitting member, the second optoelectronic element for emitted light outputs a second emitted light reception signal upon receiving a part of the ultraviolet light emitted from the second optoelectronic element, the first optoelectronic element or the third optoelectronic element outputs a second transmitted light reception signal upon receiving the ultraviolet light emitted from the second optoelectronic element and subsequently transmitted through the second light-transmitting member, the fluid, and the first light-transmitting member, and the processor controls emission of the ultraviolet light from the second optoelectronic element based on the fourth reflected light reception signal, the second emitted light reception signal, and the second transmitted light reception signal. In the fourth state, the second optoelectronic element outputs a second reflected light reception signal upon receiving the ultraviolet light emitted from the fourth optoelectronic element and reflected by the second light-transmitting member, the fourth optoelectronic element for emitted light outputs a fourth emitted light reception signal upon receiving a part of the ultraviolet light emitted from the fourth optoelectronic element, the first optoelectronic element or the third optoelectronic element outputs a fourth transmitted light reception signal upon receiving the ultraviolet light emitted from the fourth optoelectronic element and subsequently transmitted through the second light-transmitting member, the fluid, and the first light-transmitting member, and the processor controls emission of the ultraviolet light from the fourth optoelectronic element based on the second reflected light reception signal, the fourth emitted light reception signal, and the fourth transmitted light reception signal.

### BRIEF DESCRIPTION OF THE DRAWINGS

FIG. 1 is a block diagram illustrating an overall configuration of a fluid sterilizing apparatus according to a first embodiment of the present disclosure.

FIG. 2 is a schematic cross-sectional view illustrating a configuration of the fluid sterilizing apparatus in the vicinity of a chamber included in the fluid sterilizing apparatus, according to the first embodiment.

FIG. 3 is a schematic top view illustrating a configuration around a first optoelectronic element included in the fluid sterilizing apparatus according to the first embodiment.

FIG. 4 is a schematic bottom view of a light-receiving sensor unit included in the fluid sterilizing apparatus according to the first embodiment.

FIG. 5 is a block diagram illustrating an overall configuration of a fluid sterilizing apparatus according to a second embodiment of the present disclosure.

FIG. 6 is a schematic cross-sectional view illustrating a state I of the fluid sterilizing apparatus according to the second embodiment.

FIG. 7 is a schematic cross-sectional view illustrating a state II of the fluid sterilizing apparatus according to the second embodiment.

FIG. 8 is a schematic top view illustrating a configuration around a first optoelectronic element included in the fluid sterilizing apparatus according to the second embodiment.

FIG. 9 is a block diagram illustrating an overall configuration of a fluid sterilizing apparatus according to a third embodiment of the present disclosure.

FIG. 10 is a schematic cross-sectional view illustrating a configuration of the fluid sterilizing apparatus in the vicinity of a chamber included in the fluid sterilizing apparatus, according to the third embodiment.

FIG. 11 is a schematic cross-sectional view illustrating a first state of the fluid sterilizing apparatus according to the third embodiment.

FIG. 12 is a schematic cross-sectional view illustrating a second state of the fluid sterilizing apparatus according to the third embodiment.

FIG. 13 is a schematic cross-sectional view illustrating a third state of the fluid sterilizing apparatus according to the third embodiment.

FIG. 14 is a schematic cross-sectional view illustrating a fourth state of the fluid sterilizing apparatus according to the third embodiment.

FIG. 15 is a schematic top view illustrating a configuration around a first optoelectronic element and a third optoelectronic element included in the fluid sterilizing apparatus according to the third embodiment.

FIG. 16 is a block diagram illustrating an overall configuration of a fluid sterilizing apparatus according to a fourth embodiment of the present disclosure.

FIG. 17 is a schematic cross-sectional view illustrating a configuration of the fluid sterilizing apparatus in the vicinity of a chamber included in the fluid sterilizing apparatus, according to the fourth embodiment.

### DETAILED DESCRIPTION OF THE DISCLOSURE

Embodiments according to the present disclosure provide a fluid sterilizing apparatus configured to irradiate a fluid with a required dose of ultraviolet light.

Hereinafter, the embodiments of the present disclosure will be described with reference to the drawings. The drawings schematically illustrate the embodiments of the present disclosure, and thus scales, intervals, positional relationships, or the like of the members may be exaggerated, or illustration of parts of the members may be omitted. As a cross-sectional view, an end view illustrating a cutting plane alone may be used.

In the following description, components having substantially the same function are denoted by the same reference symbols, and description of the components may be omitted. Also, terms indicating specific directions or positions (e.g., “over”, “top”, “above”, “upper”, “bottom”, “below”, “lower”, and other terms including these terms) are merely used for ease of understanding of relative directions or positions in referenced drawings. As long as a relative directional or positional relationship indicated by terms, such as “top”, “bottom”, and the like, is consistent in the referenced drawings, drawings other than those of the present disclosure, actual products, and the like do not need to have an arrangement the same as those in the referenced drawings. In the present specification, the side at which the largest quantity of light is emitted from a first optoelectronic element included in a fluid sterilizing apparatus according to the embodiments of the present disclosure is referred to as “top”, and the side opposite to “top” via the first optoelectronic element is referred to as “bottom”. The term “face” means facing each other, but is not limited to direct facing. The term “face” also includes a case in which, in a cone whose axis of symmetry is a half-line perpendicular to a disposition plane of one thing and passing through the center of gravity of the one thing, whose apex is the center of gravity of the one thing, and that has an apex angle of 90 degrees (°) in a plane including the axis of symmetry, the other thing is present in this cone. The term “optoelectronic element” can include, for example, one or more light-emitting diodes, solar cells, photodiodes, laser diodes, other optoelectronic elements, and combinations of these. The term “reflected light” can include light reflected at a certain interface, light passed through any path after being reflected at a certain interface, and the like. Note that the reflected light includes at least one of specular reflected light or diffuse reflected light. The term “reflected light reception signal” refers to a signal generated from the optoelectronic element upon directly receiving reflected light or indirectly receiving reflected light. “Indirectly receiving reflected light” means that the optoelectronic element receives reflected light through an optical member, such as a light guide plate, an optical fiber, a prism, a lens, or the like. “Directly receiving reflected light” means that the optoelectronic element receives reflected light without any intervening optical member described above.

The positional relationship represented by “over” in the present specification includes a case in which things are in contact with each other, and a case in which things are not in contact with each other but one of them is positioned above the other. The term “dispose” is not limited to a case in which things are in direct contact with each other, and includes a case in which things are disposed indirectly, for example, via another member.

In the present specification or the claims, when there are a plurality of components and these components are to be distinguishably expressed, the terms “first” and “second” may be added before these components for distinction. Also, there may be a case in which objects to be distinguished in the present specification are different from objects to be distinguished in the claims.

### First Embodiment

<Configuration of Fluid Sterilizing Apparatus according to First Embodiment of Present Disclosure>

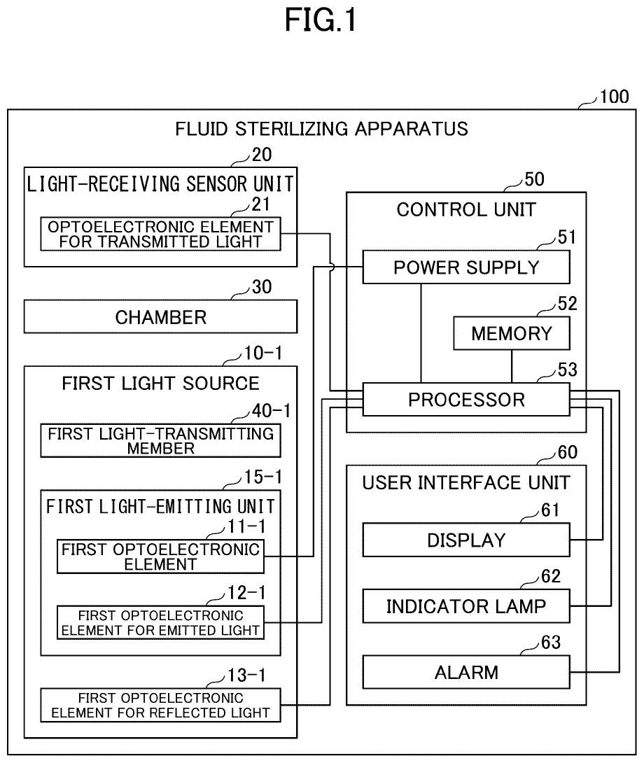

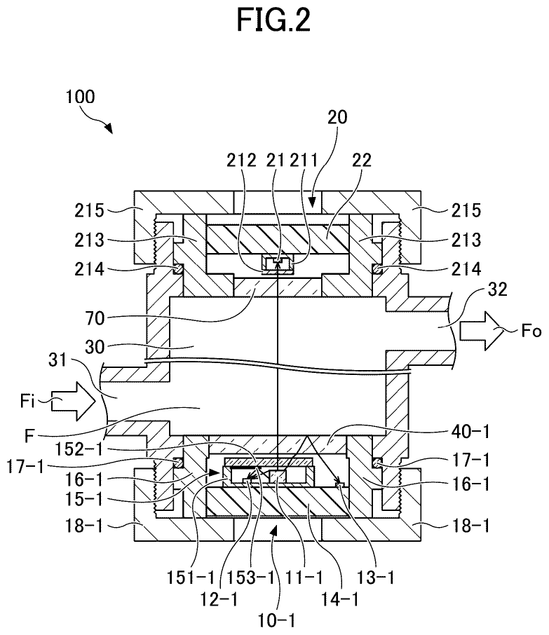

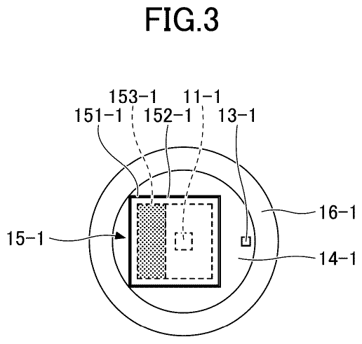

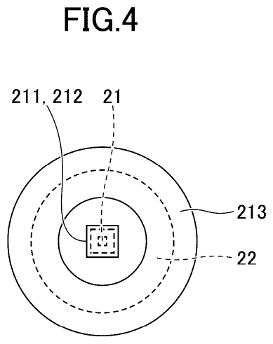

A configuration of a fluid sterilizing apparatus according to a first embodiment of the present disclosure will be described with reference to FIGS. 1 to 4. FIG. 1 is a block diagram illustrating an overall configuration of a fluid sterilizing apparatus 100 according to the first embodiment of the present disclosure. FIG. 2 is a schematic cross-sectional view illustrating a configuration of the fluid sterilizing apparatus 100 in the vicinity of a chamber 30 included in the fluid sterilizing apparatus 100. FIG. 3 is a schematic top view illustrating a configuration around the first optoelectronic element included in the fluid sterilizing apparatus 100. FIG. 4 is a schematic bottom view of a light-receiving sensor unit 20 included in the fluid sterilizing apparatus 100.

### (Overall Configuration)

As illustrated in FIGS. 1 and 2, the fluid sterilizing apparatus 100 includes a first light source 10-1, the light-receiving sensor unit 20, the chamber 30, and a control unit 50. In the example illustrated in FIGS. 1 and 2, the fluid sterilizing apparatus 100 includes a user interface unit 60. Also, the first light source 10-1 includes a first light-transmitting member 40-1. Further, the light-receiving sensor unit 20 includes a light-transmitting member for transmitted light 70. FIG. 2 illustrates a cross section of the fluid sterilizing apparatus 100 including the first light source 10-1, the light-receiving sensor unit 20, and the chamber 30.

The fluid sterilizing apparatus 100 is configured to irradiate a fluid F located in an inner space of the chamber 30 with ultraviolet light from the first light source 10-1, thereby sterilizing bacteria or viruses in the fluid F. The fluid sterilizing apparatus 100 can reduce the number of bacteria or viruses in the fluid F after irradiation with ultraviolet light compared to before irradiation with ultraviolet light.

The first light source 10-1 includes, in addition to the first light-transmitting member 40-1, a first optoelectronic element 11-1, a first optoelectronic element for emitted light 12-1, and a first optoelectronic element for reflected light 13-1. The light-receiving sensor unit 20 includes an optoelectronic element for transmitted light 21. The optoelectronic element for transmitted light 21 is disposed to face the first optoelectronic element 11-1, the first optoelectronic element for emitted light 12-1, and the first optoelectronic element for reflected light 13-1.

The chamber 30 includes the inner space that allows passage of the fluid F to be irradiated with ultraviolet light emitted from the first optoelectronic element 11-1. The fluid F located in the inner space of the chamber 30 is disposed between: the first optoelectronic element 11-1, the first optoelectronic element for emitted light 12-1, and the first optoelectronic element for reflected light 13-1; and the optoelectronic element for transmitted light 21. The fluid F located in the inner space of the chamber 30 is irradiated with ultraviolet light emitted from the first optoelectronic element 11-1.

The first light-transmitting member 40-1 is disposed between: the first optoelectronic element 11-1, the first optoelectronic element for emitted light 12-1, and the first optoelectronic element for reflected light 13-1; and the inner space of the chamber 30.

The light-transmitting member for transmitted light 70 is disposed between the optoelectronic element for transmitted light 21 and the inner space of the chamber 30.

The control unit 50 includes a processor 53 configured to control the emission of ultraviolet light from the first optoelectronic element 11-1 upon receiving a first reflected light reception signal, a first emitted light reception signal, and a first transmitted light reception signal. The first reflected light reception signal is an electrical signal generated from the first optoelectronic element for reflected light 13-1 upon receiving the ultraviolet light emitted from the first optoelectronic element 11-1 and reflected by the first light-transmitting member 40-1. The first emitted light reception signal is an electrical signal generated from the first optoelectronic element for emitted light 12-1 upon receiving a part of the ultraviolet light emitted from the first optoelectronic element 11-1. The first transmitted light reception signal is an electrical signal generated from the optoelectronic element for transmitted light 21 upon receiving the ultraviolet light emitted from the first optoelectronic element 11-1 and subsequently transmitted through the first light-transmitting member 40-1 and the fluid F. The electrical signals from the optoelectronic elements, such as the first optoelectronic element for reflected light 13-1, the first optoelectronic element for emitted light 12-1, and the optoelectronic element for transmitted light 21, are analog current signals or analog voltage signals.

In a typical fluid sterilizing apparatus, application of a predetermined dose of ultraviolet light is required for sterilization of a fluid, as required by, for example, NSF/ANSI 55-2024. The dose of ultraviolet light is a total quantity of energy applied through irradiation with ultraviolet light, and is expressed as a product of an illuminance and an irradiation period. On the other hand, the lifetime of a light source included in the typical fluid sterilizing apparatus decreases as the drive current increases in accordance with an output of light from the light source in proportion to the illuminance. As the light source becomes closer to the end of lifetime, the drive current increases, necessitating more frequent maintenance, such as, for example, cleaning to remove contaminants. If the removal of contaminants is not appropriately performed, the light source breaks down, and thus maintenance, such as, for example, replacement of the light source is needed. That is, maintainability of the fluid sterilizing apparatus is impaired. Therefore, for maintaining sterilization performance without impairing maintainability, it is required to stably irradiate a fluid with ultraviolet light at an illuminance that is required for obtaining the predetermined dose of ultraviolet light. It is required to limit a drive current for obtaining an illuminance that is higher than necessary.

As a typical method of irradiating a fluid with a required dose of ultraviolet light, a method conceivable when a flow rate of the fluid is substantially constant includes receiving ultraviolet light emitted from a light source by a light-receiving sensor, and feedback-controlling a drive current of the light source. This is because a period during which the fluid is irradiated with ultraviolet light in a chamber is inversely proportional to the flow rate. In a typical fluid sterilizing apparatus, there may be a case in which contaminants, such as slime, mold, water stain, and the like, adheres, for example, to an inner wall of the chamber included in the fluid sterilizing apparatus. If the contaminants adhere to the light-transmitting member disposed between the fluid in the chamber and the light source, the contaminants can absorb or scatter the ultraviolet light from the light source, thereby reducing the dose of ultraviolet light applied to the fluid. Also, if the contaminants adhere to the light-transmitting member disposed between the fluid in the chamber and the light-receiving sensor, the contaminants can absorb or scatter the ultraviolet light received by the light-receiving sensor, thereby lowering accuracy of the feedback control of the dose of ultraviolet light. As a result, there is a possibility that the fluid cannot be stably irradiated with a required dose of ultraviolet light.

In the fluid sterilizing apparatus 100 according to the first embodiment of the present disclosure, the control unit 50 includes the processor 53 configured to receive the first emitted light reception signal including information of the output of light emitted from the first optoelectronic element 11-1. Also, the control unit 50 includes the processor 53 configured to receive the first reflected light reception signal that accounts for effects, such as light absorption, scattering, or the like, caused by at least one of the first light-transmitting member 40-1 or the contaminants adhering to the first light-transmitting member 40-1. Further, the control unit 50 includes the processor 53 configured to receive the first transmitted light reception signal that accounts for effects, such as light absorption, scattering, or the like, caused by at least one of the first light-transmitting member 40-1, the contaminants adhering to the first light-transmitting member 40-1, the fluid F, the light-transmitting member for transmitted light 70, or the contaminants adhering to the light-transmitting member for transmitted light 70.

The control unit 50 includes the processor 53 configured to control the emission of the ultraviolet light from the first optoelectronic element 11-1 upon receiving the first reflected light reception signal, the first emitted light reception signal, and the first transmitted light reception signal, i.e., by mitigating the impact of the contaminants adhering to the first light-transmitting member 40-1 and the light-transmitting member for transmitted light 70. For example, the control unit 50 included in the fluid sterilizing apparatus 100 includes the processor 53 configured to control the dose of ultraviolet light applied to the fluid F from the first optoelectronic element 11-1 to be within a predetermined range by adjusting the drive current supplied to the first optoelectronic element 11-1. This predetermined range is, for example, within ±5%, preferably within ±3%, and more preferably within ±1% of the specification about the dose of ultraviolet light.

The fluid sterilizing apparatus 100 can irradiate the fluid with the required dose of ultraviolet light by controlling the emission of ultraviolet light from the first optoelectronic element 11-1 while mitigating the impact of the contaminants adhering to the first light-transmitting member 40-1 and the light-transmitting member for transmitted light 70. As a result, the fluid sterilizing apparatus 100 can improve maintainability while maintaining sterilization performance. Irradiating the fluid with the required dose of ultraviolet light means, for example, making constant the illuminance of ultraviolet light applied to the fluid F flowing at a constant flow rate, thereby making constant the dose of ultraviolet light. The recitation “the dose of ultraviolet light is constant” includes the dose of ultraviolet light being within ±5%, preferably within ±3%, and more preferably within ±1% of the specification about the dose of ultraviolet light. Details of the effects of the fluid sterilizing apparatus 100 will be described below in the description of the operations and effects of the fluid sterilizing apparatus 100.

In the following description, an example in which the fluid is a liquid will be described. However, the fluid may be a gas. Further, the following description is for the fluid sterilizing apparatus 100 in which the flow rate of the fluid is constant.

### (First Light Source 10-1)

In the example illustrated in FIGS. 2 and 3, the first light source 10-1 includes a first substrate 14-1, a first light-emitting unit 15-1 disposed over the first substrate 14-1, a first light-transmitting member 40-1, and a first frame 16-1 that supports the first substrate 14-1 and the first light-transmitting member 40-1. The first light source 10-1 includes a first sealing member 17-1 disposed between the chamber 30 and the first frame 16-1, and a first fixing member 18-1 that fixes the first light source 10-1 to the chamber 30.

In the example illustrated in FIG. 3, the first substrate 14-1 has an outer peripheral shape that is substantially circular in a top view. The first frame 16-1 has a hollow circular shape in a top view, and the first substrate 14-1 is disposed in the inner space of the first frame 16-1. However, the first substrate 14-1 may have an outer peripheral shape that is substantially rectangular, substantially elliptical, substantially polygonal, or the like in a top view. The first frame 16-1 may have a hollow rectangular shape in a top view, and the first substrate 14-1 may be disposed in the inner space of the first frame 16-1. As a material of the first frame 16-1, a metal, such as SUS or the like, or an engineering plastic may be used.

The first light source 10-1 may include a plurality of first light-emitting units 15-1. Also, the first light source 10-1 may include the first light-emitting unit 15-1 including the first optoelectronic element for emitted light 12-1, and the first light-emitting unit 15-1 not including the first optoelectronic element for emitted light 12-1.

The first light source 10-1 can be removed from the fluid sterilizing apparatus 100. The ability to remove the first light source 10-1 from the fluid sterilizing apparatus 100 facilitates removal of the contaminants adhering to the first light-transmitting member 40-1, replacement of the first substrate 14-1 over which the first light-emitting unit 15-1 is placed, or the like. The first sealing member 17-1 ensures water tightness between the first light source 10-1 and the inner space of the chamber 30.

An internal thread is formed at the inner surface of the first fixing member 18-1. The internal thread of the first fixing member 18-1 is screw-connected with an external thread formed at a portion of the chamber 30 corresponding to the internal thread. By screwing and tightening the first fixing member 18-1 and pressing the first frame 16-1 against the chamber 30, the first light source 10-1 can be attached and fixed to the chamber 30. The first fixing member 18-1 can be removed from the chamber 30 by loosening the screw connection between the internal thread of the first fixing member 18-1 and the external thread of the chamber 30. By removing the first fixing member 18-1 from the chamber 30, the first light source 10-1 can be removed from the chamber 30. However, the configuration and method for attaching and removing the first light source 10-1 relative to the fluid sterilizing apparatus 100 are not limited to the above, and can be appropriately changed.

The first light-transmitting member 40-1 may be separately provided without being included in the first light source 10-1. When the first light-transmitting member 40-1 is provided separately from the first light source 10-1, and maintenance of the first light-transmitting member 40-1, such as cleaning, replacement, or the like, is to be performed, the first light-transmitting member 40-1 can be removed from the fluid sterilizing apparatus 100 separately from or along with the first light source 10-1, and the maintenance of the first light-transmitting member 40-1 can be performed separately from the first light source 10-1.

### (First Optoelectronic Element 11-1)

The first optoelectronic element 11-1 is an element configured to emit ultraviolet light having a bactericidal ability or an inactivating ability against bacteria or viruses contained in the fluid F. The first optoelectronic element 11-1 is, for example, an UV-LED (Ultraviolet-Light Emitting Diode) chip. However, the first optoelectronic element 11-1 may be an UV-LD (Ultraviolet-Laser Diode) chip. The first optoelectronic element 11-1 is disposed to face the first light-transmitting member 40-1.

The peak wavelength of the ultraviolet light emitted by the first optoelectronic element 11-1 is, for example, 200 nanometers (nm) to 400 nanometers (nm). The first optoelectronic element 11-1 includes, for example, a light-transmitting substrate for growth, a multilayer film of a Group III nitride mixed crystal semiconductor, and positive and negative electrodes. The first optoelectronic element 11-1 includes an n-side semiconductor layer to be electrically connected to the negative electrode, a p-side semiconductor layer to be electrically connected to the positive electrode, and an active layer between the n-side semiconductor layer and the p-side semiconductor layer. The first optoelectronic element 11-1 may be electrically connected, via a wire, to an internal electrode terminal of a first housing 151-1 of the first light-emitting unit 15-1, or may be electrically connected, through flip-chip connection, to the internal electrode terminal of the first housing 151-1. One or more first optoelectronic elements 11-1 can be disposed in the single first housing 151-1.

### (First Optoelectronic Element for Emitted Light 12-1)

The first optoelectronic element for emitted light 12-1 is an element configured to directly or indirectly receive a part of the ultraviolet light emitted from the first optoelectronic element 11-1, and output, to the control unit 50, the first emitted light reception signal in accordance with the illuminance of the received ultraviolet light. The first emitted light reception signal is a photocurrent or photovoltage. The first emitted light reception signal includes information of the output of light emitted from the first optoelectronic element 11-1.

“Directly receiving light” means that the first optoelectronic element for emitted light 12-1 receives a part of the ultraviolet light emitted from the first optoelectronic element 11-1 without any intervening member other than the first optoelectronic element for emitted light 12-1. “Indirectly receiving light” means that the first optoelectronic element for emitted light 12-1 receives a part of light emitted from the first optoelectronic element 11-1 and then reflected by a member other than the first optoelectronic element for emitted light 12-1, such as a first cover 152-1 or the like. In the example illustrated in FIG. 2, the first optoelectronic element for emitted light 12-1 directly receives a part of the ultraviolet light emitted from the first optoelectronic element 11-1, and indirectly receives another part of the ultraviolet light emitted from the first optoelectronic element 11-1 via the first cover 152-1.

A photodiode of Si, SiC, GaN, or the like can be used as the first optoelectronic element for emitted light 12-1. In this case, a bias voltage to be applied to the photodiode is appropriately selected from a zero bias and a reverse bias. At least one first optoelectronic element for emitted light 12-1 can be disposed in the single first housing 151-1.

### (First Optoelectronic Element for Reflected Light 13-1)

The first optoelectronic element for reflected light 13-1 is an element configured to receive, of ultraviolet light emitted from the first light-emitting unit 15-1, the ultraviolet light reflected by the first light-transmitting member 40-1, and output, to the control unit 50, the first reflected light reception signal in accordance with the illuminance of the received ultraviolet light. For example, the first optoelectronic element for reflected light 13-1 receives, of the ultraviolet light emitted from the first light-emitting unit 15-1, the ultraviolet light reflected at the interface between the first light-transmitting member 40-1 and the fluid F. The first reflected light reception signal is a photocurrent or photovoltage. The first reflected light reception signal accounts for effects, such as light absorption, scattering, or the like, caused by at least one of the first cover 152-1, the first light-transmitting member 40-1, or the contaminants adhering to the first light-transmitting member 40-1.

A photodiode of Si, SiC, GaN, or the like can be used as the first optoelectronic element for reflected light 13-1. In this case, a bias voltage to be applied to the photodiode is appropriately selected from a zero bias and a reverse bias. The first optoelectronic element for reflected light 13-1 is disposed to face the first light-transmitting member 40-1.

When the first optoelectronic element for reflected light 13-1 is disposed in a space that is airtight, a housing-equipped photodiode or a photodiode chip can be used as the first optoelectronic element for reflected light 13-1. When the first optoelectronic element for reflected light 13-1 is disposed in a space that is not airtight, a housing-equipped photodiode, which readily ensures airtightness, is preferably used as the first optoelectronic element for reflected light 13-1 from the viewpoint of ensuring reliability.

### (First Substrate 14-1)

The first substrate 14-1 is a wiring board including a base of an insulator, a region to which the first light-emitting unit 15-1 is to be connected, a region to which the first optoelectronic element for reflected light 13-1 is to be connected, and predetermined conductive structures leading to these regions. In the example illustrated in FIG. 2, the region to which the first light-emitting unit 15-1 is to be connected and the region to which the first optoelectronic element for reflected light 13-1 is to be connected directly face the first light-transmitting member 40-1, and the first optoelectronic element 11-1 and the first optoelectronic element for reflected light 13-1 are disposed to directly face the first light-transmitting member 40-1. When the first substrate 14-1 includes conductive structures to which the plurality of first light-emitting units 15-1 are to be connected, the plurality of first light-emitting units 15-1 may be connected in series, in parallel, or both in series and in parallel.

A detachable connecting terminal configured to electrically connect external conductive structures to conductive structures over the first substrate 14-1 may be disposed at a first surface closer to the fluid F in the first substrate 14-1, a second surface opposite to the first surface, or both the first surface and the second surface. The first substrate 14-1 may include a substrate including a region to which the first light-emitting unit 15-1 is to be connected, and a substrate including a region to which the first optoelectronic element for reflected light 13-1 is to be connected. Also, the first substrate 14-1 may include an annular wall in a top view, and the surface of an interior enclosed by the annular wall may be provided with a region to which the first light-emitting unit 15-1 is to be connected and a region to which the first optoelectronic element for reflected light 13-1 is to be connected. In this case, the region to which the first optoelectronic element for reflected light 13-1 is to be connected may be disposed at the inner surface of the above wall, which does not directly face the first light-transmitting member 40-1.

The first substrate 14-1 is fixed to the first frame 16-1 through screwing, clipping, or the like, and thus can be readily removed from the first frame 16-1.

### (First Light-Emitting Unit 15-1)

In the example illustrated in FIG. 2, the first light-emitting unit 15-1 includes a first housing 151-1 and a first cover 152-1 in addition to the first optoelectronic element 11-1 and the first optoelectronic element for emitted light 12-1.

### (First Housing 151-1 and First Cover 152-1)

The first cover 152-1 is disposed to face the first optoelectronic element 11-1 and disposed between the first optoelectronic element 11-1 and the first light-transmitting member 40-1. The first cover 152-1 reflects a part of the ultraviolet light emitted from the first optoelectronic element 11-1, and transmits all or a part of the remainder. The first optoelectronic element for emitted light 12-1 is configured to receive a part of the ultraviolet light reflected by the first cover 152-1.

The first housing 151-1 houses the first optoelectronic element 11-1 and the first optoelectronic element for emitted light 12-1, and supports the first cover 152-1.

The first housing 151-1 and the first cover 152-1 hermetically seal the first optoelectronic element 11-1 and the first optoelectronic element for emitted light 12-1 from the exterior of the first light-emitting unit 15-1. For the first housing 151-1 and the first cover 152-1, a ceramic package, including a ceramic member or a metal-ceramic composite member, or a CAN package can be used. In the example illustrated in FIG. 3, the first housing 151-1 and the first cover 152-1 have an outer peripheral shape that is substantially rectangular in a top view. However, the first housing 151-1 and the first cover 152-1 may have an outer peripheral shape that is substantially circular, substantially elliptical, or substantially polygonal in a top view.

The first housing 151-1 includes an external electrode terminal to be electrically connected to the conductive structures of the first substrate 14-1, an internal electrode terminal to be electrically connected to the first optoelectronic element 11-1, an internal electrode terminal to be electrically connected to the first optoelectronic element for emitted light 12-1, and a conductive member configured to electrically connect the external electrode terminal to the internal electrode terminal. As the first housing 151-1, a metal-ceramic composite member or the like can be used.

The first cover 152-1 includes a member having light transmittivity. The light transmittivity of the light-transmitting surface of the first cover 152-1 preferably exhibits a light transmittance that allows 60% or more of the ultraviolet light emitted from the first optoelectronic element 11-1 to pass through. The first cover 152-1 transmits a part of the ultraviolet light emitted from the first optoelectronic element 11-1, and reflects all or a part of the remainder.

The first cover 152-1 includes a window portion that transmits a part of the ultraviolet light emitted from the first optoelectronic element 11-1, and a support that supports the window portion and is joined with the first housing 151-1. The window portion of the first cover 152-1 may include an optical thin film (AR coat) that increases the transmittance of the ultraviolet light emitted from the first optoelectronic element 11-1. The first cover 152-1 may have an optical element function, such as a lens function.

As a material of the window portion of the first cover 152-1, for example, it is possible to use an inorganic material formed of at least one selected from the group consisting of quartz glass, borosilicate glass, calcium fluoride glass, aluminoborosilicate glass, oxynitride glass, chalcogenide glass, and sapphire. The window portion included in the first cover 152-1 may be joined with the support included in the first cover 152-1 by use of low-melting-point glass that mainly includes lead and oxygen.

In the example illustrated in FIGS. 2 and 3, the first cover 152-1 includes a first light quantity adjusting member 153-1. The first light quantity adjusting member 153-1 is disposed to face the first optoelectronic element for emitted light 12-1. Specifically, the first light quantity adjusting member 153-1 is provided at a surface of the first cover 152-1 on the first optoelectronic element 11-1 side and at a position not overlapping the first optoelectronic element 11-1 in a top view. The first light quantity adjusting member 153-1 has an outer peripheral shape that is substantially rectangular in a top view. However, the first light quantity adjusting member 153-1 may have a shape that is substantially square, substantially circular, substantially elliptical, annular, or the like in a top view.

The first light quantity adjusting member 153-1 reduces light incident on the first optoelectronic element for emitted light 12-1, housed in the first housing 151-1, from the exterior of the first housing 151-1, and reflects the ultraviolet light emitted from the first optoelectronic element 11-1. The first light quantity adjusting member 153-1 suppresses reduction in control accuracy of the quantity of the ultraviolet light emitted from the first optoelectronic element 11-1 due to light from the exterior of the first light-emitting unit 15-1.

The first light quantity adjusting member 153-1 is, for example, a multilayer film having a double-layer structure of an aluminum layer or a metallic chromium layer and a chromium oxide layer, or a dielectric multilayer film.

### (First Light-Transmitting Member 40-1)

The first light-transmitting member 40-1 can be a flat-plate member having light transmittivity. The light transmittivity of the first light-transmitting member 40-1 preferably exhibits a light transmittance that allows 60% or more of the ultraviolet light emitted from the first optoelectronic element 11-1 to pass through. The first light-transmitting member 40-1 may have a differently shaped plane other than a flat plane, such as a curved plane or the like, in at least a part of the first light-transmitting member 40-1. The first light-transmitting member 40-1 may include a lens, a prism, a light guide structure, or the like. From the viewpoint of facilitating removal of contaminants adhering to the first light-transmitting member 40-1, a plane of the first light-transmitting member 40-1 in contact with the fluid F is preferably a flat plane.

In the example illustrated in FIG. 2, the first light-transmitting member 40-1 is disposed between: the first substrate 14-1, the first light-emitting unit 15-1, and the first optoelectronic element for reflected light 13-1; and the inner space of the chamber 30. By the presence of the first light-transmitting member 40-1, the fluid F does not contact the first substrate 14-1, the first light-emitting unit 15-1, and the first optoelectronic element for reflected light 13-1.

The first light-transmitting member 40-1 transmits a part of the ultraviolet light emitted from the first light-emitting unit 15-1, and reflects all or a part of the remainder. As a material of the first light-transmitting member 40-1, for example, it is possible to use an inorganic material formed of at least one selected from the group consisting of quartz glass, borosilicate glass, calcium fluoride glass, aluminoborosilicate glass, oxynitride glass, chalcogenide glass, quartz, and sapphire. Also, the first light-transmitting member 40-1 may include an optical thin film at a light-transmitting surface of the first light-transmitting member 40-1 for adjustment of transmittance and reflectance.

(Light-Receiving Sensor Unit 20)

In the example illustrated in FIG. 2, the light-receiving sensor unit 20 includes a substrate for transmitted light 22, a housing for transmitted light 211 disposed below the substrate for transmitted light 22, and a cover for transmitted light 212 disposed between the optoelectronic element for transmitted light 21 and the light-transmitting member for transmitted light 70. Also, the light-receiving sensor unit 20 includes the light-transmitting member for transmitted light 70, a frame for transmitted light 213 that supports the substrate for transmitted light 22 and the light-transmitting member for transmitted light 70, a sealing member for transmitted light 214 disposed between the chamber 30 and the frame for transmitted light 213, and a fixing member for transmitted light 215 that fixes the light-receiving sensor unit 20 to the chamber 30. The optoelectronic element for transmitted light 21 is housed in the housing for transmitted light 211.

In the example illustrated in FIG. 4, the substrate for transmitted light 22 has an outer peripheral shape that is substantially circular in a bottom view. The frame for transmitted light 213 has a hollow circular shape in a bottom view, and the substrate for transmitted light 22 is disposed in the inner space of the frame for transmitted light 213. However, the substrate for transmitted light 22 may have an outer peripheral shape that is substantially rectangular, substantially elliptical, substantially polygonal, or the like in a bottom view. The frame for transmitted light 213 may have a hollow rectangular shape in a bottom view, and the substrate for transmitted light 22 may be disposed in the inner space of the frame for transmitted light 213.

The light-receiving sensor unit 20 can be removed from the fluid sterilizing apparatus 100. The ability to remove the light-receiving sensor unit 20 from the fluid sterilizing apparatus 100 facilitates removal of the contaminants adhering to the light-transmitting member for transmitted light 70, replacement of the substrate for transmitted light 22 over which the optoelectronic element for transmitted light 21 is placed, or the like. The sealing member for transmitted light 214 ensures water tightness between the light-receiving sensor unit 20 and the inner space of the chamber 30.

An internal thread is formed at the inner surface of the fixing member for transmitted light 215. The internal thread of the fixing member for transmitted light 215 is screw-connected with an external thread formed at a portion of the chamber 30 corresponding to the internal thread. By screwing and tightening the fixing member for transmitted light 215 and pressing the frame for transmitted light 213 against the chamber 30, the light-receiving sensor unit 20 can be attached and fixed to the chamber 30. The fixing member for transmitted light 215 can be removed from the chamber 30 by loosening the screw connection between the internal thread of the fixing member for transmitted light 215 and the external thread of the chamber 30. By removing the fixing member for transmitted light 215 from the chamber 30, the light-receiving sensor unit 20 can be removed from the chamber 30. However, the configuration and method for attaching and removing the light-receiving sensor unit 20 relative to the fluid sterilizing apparatus 100 are not limited to the above, and can be appropriately changed.

The light-transmitting member for transmitted light 70 may be separately provided without being included in the light-receiving sensor unit 20. When the light-transmitting member for transmitted light 70 is provided separately from the light-receiving sensor unit 20, and maintenance of the light-transmitting member for transmitted light 70, such as cleaning, replacement, or the like, is to be performed, the light-transmitting member for transmitted light 70 can be removed from the fluid sterilizing apparatus 100 separately from or along with the light-receiving sensor unit 20, and the maintenance of the light-transmitting member for transmitted light 70 can be performed separately from the light-receiving sensor unit 20.

### (Optoelectronic Element for Transmitted Light 21)

The optoelectronic element for transmitted light 21 is an element configured to receive a part of the ultraviolet light emitted from the first optoelectronic element 11-1 and subsequently transmitted through the first cover 152-1, the first light-transmitting member 40-1, the fluid F located in the inner space of the chamber 30, the light-transmitting member for transmitted light 70, and the cover for transmitted light 212. The optoelectronic element for transmitted light 21 outputs, to the control unit 50, the first transmitted light reception signal in accordance with the illuminance of the received ultraviolet light. The first transmitted light reception signal is a photocurrent or photovoltage. The first transmitted light reception signal accounts for effects, such as light absorption, scattering, or the like, caused by at least one of the first cover 152-1, the first light-transmitting member 40-1, the contaminants adhering to the first light-transmitting member 40-1, the fluid F, the light-transmitting member for transmitted light 70, or the cover for transmitted light 212. A photodiode of Si, SiC, GaN, or the like can be used as the optoelectronic element for transmitted light 21. In this case, a bias voltage to be applied to the photodiode is appropriately selected from a zero bias and a reverse bias.

(Housing for Transmitted Light 211 and Cover for Transmitted Light 212)

The cover for transmitted light 212 is disposed to face the optoelectronic element for transmitted light 21 and disposed between the optoelectronic element for transmitted light 21 and the light-transmitting member for transmitted light 70. The cover for transmitted light 212 transmits a part of the ultraviolet light emitted from the first optoelectronic element 11-1. The optoelectronic element for transmitted light 21 receives the ultraviolet light emitted from the first optoelectronic element 11-1 and subsequently transmitted through the cover for transmitted light 212.

The housing for transmitted light 211 is a member that houses the optoelectronic element for transmitted light 21 and supports the cover for transmitted light 212.

The housing for transmitted light 211 and the cover for transmitted light 212 hermetically seal the optoelectronic element for transmitted light 21, housed in the housing for transmitted light 211, from the exterior of the housing for transmitted light 211 and the cover for transmitted light 212. For the housing for transmitted light 211 and the cover for transmitted light 212, a ceramic package, including a ceramic member or a metal-ceramic composite member, or a CAN package can be used.

In the example illustrated in FIG. 4, the housing for transmitted light 211 and the cover for transmitted light 212 have a substantially rectangular outer peripheral shape in a bottom view. The housing for transmitted light 211 and the cover for transmitted light 212 may have an outer peripheral shape that is substantially circular, substantially elliptical, or substantially polygonal in a bottom view. The housing for transmitted light 211 and the cover for transmitted light 212 overlap with each other in a bottom view. Thus, in FIG. 4, the reference numerals of the housing for transmitted light 211 and the cover for transmitted light 212 are described side by side. In the subsequent drawings, there may be a case in which some reference numerals are described side by side for the same reason.

The housing for transmitted light 211 includes an external electrode terminal to be electrically connected to the conductive structures of the substrate for transmitted light 22, an internal electrode terminal to be electrically connected to the optoelectronic element for transmitted light 21, and a conductive member configured to electrically connect the external electrode terminal to the internal electrode terminal. As the housing for transmitted light 211, a metal-ceramic composite member or the like can be used.

The cover for transmitted light 212 includes a member having light transmittivity. The light transmittivity of the light-transmitting surface of the cover for transmitted light 212 preferably exhibits a light transmittance that allows 60% or more of the ultraviolet light emitted from the first optoelectronic element 11-1 to pass through.

The cover for transmitted light 212 includes a window portion that transmits a part of the ultraviolet light emitted from the first optoelectronic element 11-1, and a support that supports the window portion and is joined with the cover for transmitted light 212. The window portion of the cover for transmitted light 212 may include an optical thin film (AR coat) that increases the transmittance of the ultraviolet light emitted from the first optoelectronic element 11-1. The cover for transmitted light 212 may have an optical element function, such as a lens function.

As a material of the window portion of the cover for transmitted light 212, for example, it is possible to use an inorganic material formed of at least one selected from the group consisting of quartz glass, borosilicate glass, calcium fluoride glass, aluminoborosilicate glass, oxynitride glass, chalcogenide glass, and sapphire. The window portion included in the cover for transmitted light 212 may be joined with the support included in the cover for transmitted light 212 by use of low-melting-point glass that mainly includes lead oxide.

### (Substrate for Transmitted Light 22)

The substrate for transmitted light 22 is a wiring substrate including a base of an insulator, a region to which the light-receiving sensor unit 20 is to be connected, and predetermined conductive structures leading to this region.

A detachable connecting terminal configured to electrically connect external conductive structures to conductive structures below the substrate for transmitted light 22 may be disposed at a first surface closer to the fluid F in the substrate for transmitted light 22, a second surface opposite to the first surface, or both the first surface and the second surface. Also, the substrate for transmitted light 22 may include an annular wall in a bottom view, and the surface of an interior enclosed by the annular wall may be provided with a region to which the light-receiving sensor unit 20 is to be connected. The substrate for transmitted light 22 is fixed to the frame for transmitted light 213 through screwing, clipping, or the like, and thus can be readily removed from the frame for transmitted light 213.

### (Light-Transmitting Member for Transmitted Light 70)

The light-transmitting member for transmitted light 70 is a flat-plate member having light transmittivity. The light transmittivity of the light-transmitting member for transmitted light 70 preferably exhibits a light transmittance that allows 60% or more of the ultraviolet light emitted from the first optoelectronic element 11-1 to pass through. The light-transmitting member for transmitted light 70 may have a differently shaped plane other than a flat plane, such as a curved plane or the like, in at least a part of the light-transmitting member for transmitted light 70. The light-transmitting member for transmitted light 70 may include a lens, a prism, a light guide structure, or the like. From the viewpoint of facilitating removal of contaminants adhering to the light-transmitting member for transmitted light 70, a plane of the light-transmitting member for transmitted light 70 in contact with the fluid F is preferably a flat plane.

In the example illustrated in FIG. 2, the light-transmitting member for transmitted light 70 is disposed between: the substrate for transmitted light 22, the housing for transmitted light 211, and the cover for transmitted light 212; and the inner space of the chamber 30. By the presence of the light-transmitting member for transmitted light 70, the fluid F does not contact the substrate for transmitted light 22, the housing for transmitted light 211, and the cover for transmitted light 212.

The light-transmitting member for transmitted light 70 transmits a part of the ultraviolet light emitted from the first light-emitting unit 15-1 and subsequently transmitted through the transmitted light-transmitting member 40-1 and the fluid F, and the light-transmitting member for transmitted light 70 reflects another part of the ultraviolet light. As a material of the light-transmitting member for transmitted light 70, for example, it is possible to use an inorganic material formed of at least one selected from the group consisting of quartz glass, borosilicate glass, calcium fluoride glass, aluminoborosilicate glass, oxynitride glass, chalcogenide glass, quartz, and sapphire. Also, the light-transmitting member for transmitted light 70 may include an optical thin film at a light-transmitting surface of the light-transmitting member for transmitted light 70 for adjustment of transmittance and reflectance.

### (Chamber 30)

The chamber 30 includes an inlet 31 for the fluid F and an outlet 32 for the fluid F. The chamber 30 is a part of the flow path. An arrow Fi illustrated in FIG. 2 represents the fluid F flowing in the inlet 31. An arrow Fo represents the fluid F flowing out from the outlet 32. The boundary between the chamber 30 and the fluid F includes an inner wall of the chamber 30, the first light-transmitting member 40-1, and the light-transmitting member for transmitted light 70. The tubular wall of the inner wall of the chamber 30 may be coated with a PTFE film, an aluminum film, or an optical thin film for increasing the reflectance of ultraviolet light.

### (Control Unit 50)

In the example illustrated in FIG. 1, the control unit 50 includes a power supply 51 configured to supply a drive current to the first optoelectronic element 11-1, and a memory 52 that stores at least an initial value of the first transmitted light reception signal (hereinafter may be referred to as a “first transmitted light reception signal initial value”). Also, the control unit 50 includes the processor 53 configured to receive the first reflected light reception signal, the first emitted light reception signal, and the first transmitted light reception signal, cause the received signals to undergo signal processing, and adjust the drive current of the first optoelectronic element 11-1.

The memory 52 can store an initial value of the first emitted light reception signal (hereinafter may be referred to as a “first emitted light reception signal initial value”), an initial value of the first reflected light reception signal (hereinafter may be referred to as a “first reflected light reception signal initial value”), and the first transmitted light reception signal initial value. The first emitted light reception signal initial value is, for example, a magnitude of the first emitted light reception signal in a state in which no contaminant adheres to the first light-transmitting member 40-1. The first reflected light reception signal initial value is, for example, a magnitude of the first reflected light reception signal in a state in which no contaminant adheres to the first light-transmitting member 40-1. The first transmitted light reception signal initial value is, for example, a magnitude of the first transmitted light reception signal in a state in which no contaminant adheres to the first light-transmitting member 40-1 and the light-transmitting member for transmitted light 70.

The processor 53 can execute various processes for realizing the functions of the fluid sterilizing apparatus 100 by an electronic circuit configured to execute instruction codes stored in a memory. The processor 53 is, for example, a central processing unit (CPU). However, the processor 53 may be an electronic circuit other than a CPU, such as a field programmable gate array (FPGA), an application specific integrated circuit (ASIC), or the like, which is designed for specific applications.

The control unit 50 can output, to the user interface unit 60, information of a state of the fluid sterilizing apparatus 100. For example, the information of the state of the fluid sterilizing apparatus 100 includes a first error signal indicating that the first light-transmitting member 40-1 or the light-transmitting member for transmitted light 70 is contaminated, and a second error signal indicating degradation in the first optoelectronic element 11-1.

### (User Interface Unit 60)

The user interface unit 60 is a unit configured to receive an input of an operation from an operator of the fluid sterilizing apparatus 100, and output information or a signal that is recognizable by the operator. The user interface unit 60 is communicably connected to the control unit 50.

In the example illustrated in FIG. 1, the user interface unit 60 includes a display 61 configured to display information, such as a text message or the like, and an indicator lamp 62 and an alarm 63 configured to notify information of the state of the fluid sterilizing apparatus 100.

The user interface unit 60 can light the indicator lamp 62, sound the alarm 63, or display a warning message on the display 61, upon receiving a first error signal or a second error signal that is output from the control unit 50.

### <Operations and Effects of Fluid Sterilizing Apparatus 100>

Next, operations of the fluid sterilizing apparatus 100, and effects of the fluid sterilizing apparatus 100 other than those described above will be described.

In the fluid sterilizing apparatus 100, the illuminance of the ultraviolet light received by the light-receiving sensor unit 20 is dependent on the states of the first cover 152-1, the first light-transmitting member 40-1, the contaminants adhering to the first light-transmitting member 40-1, the fluid F, the contaminants adhering to the light-transmitting member for transmitted light 70, the light-transmitting member for transmitted light 70, and the cover for transmitted light 212. Also, the dose of the ultraviolet light applied to the fluid F is dependent on the states of the first cover 152-1, the first light-transmitting member 40-1, and the contaminants adhering to the first light-transmitting member 40-1. In the fluid sterilizing apparatus 100, there may be a case in which, when contaminants adhere and accumulate onto the first light-transmitting member 40-1 and the light-transmitting member for transmitted light 70, a predetermined dose of ultraviolet light required for sterilization of the fluid F cannot be ensured.

Also, when one type of contaminants adheres and accumulates onto the first light-transmitting member 40-1 or the light-transmitting member for transmitted light 70, the magnitude of the first transmitted light reception signal output from the optoelectronic element for transmitted light 21 may decrease even if the dose of ultraviolet light applied to the fluid F is constant. In this case, by increasing the drive current supplied to the first light source 10-1, it is possible to return the magnitude of the first transmitted light reception signal to the magnitude before the contaminant adheres and accumulates onto the first light-transmitting member 40-1 or the light-transmitting member for transmitted light 70. However, the dose of ultraviolet light applied to the fluid F becomes more than the dose of ultraviolet light required for sterilization of the fluid F, and thus the lifetime of the first light source 10-1 can unexpectedly become shorter in accordance with the drive current supplied to the first light source 10-1.

Further, when another type of contaminants adheres and accumulates onto the first light-transmitting member 40-1 or the light-transmitting member for transmitted light 70, the magnitude of the first transmitted light reception signal may increase even if the dose of ultraviolet light applied to the fluid F is constant. In this case, by decreasing the drive current supplied to the first light source 10-1, it is possible to return the magnitude of the first transmitted light reception signal to the magnitude before the contaminant adheres and accumulates onto the first light-transmitting member 40-1 or the light-transmitting member for transmitted light 70. However, the dose of ultraviolet light applied to the fluid F becomes less than the dose of ultraviolet light required for sterilization of the fluid F, and thus sterilization performance cannot be maintained. Therefore, there is a need to remove the contaminants adhering to the light-transmitting member for transmitted light 70, or perform calibrations, such as, for example, adjusting the amplification factor of the first transmitted light reception signal.

In addition, the magnitude of the first transmitted light reception signal may decrease due to degradation in the first optoelectronic element 11-1. In this case, replacement of the first optoelectronic element 11-1 is needed.

As described above, the factors that cause a decrease in the magnitude of the first transmitted light reception signal in the fluid sterilizing apparatus 100 are the state of contaminants adhering to the first light-transmitting member 40-1 or the light-transmitting member for transmitted light 70, and the degraded state of the first optoelectronic element 11-1. However, it is not possible, from the first transmitted light reception signal alone, to determine which of the factors causes a decrease in the magnitude of the first transmitted light reception signal: the state of contaminants adhering to the first light-transmitting member 40-1 or the light-transmitting member for transmitted light 70; or the degraded state of the first optoelectronic element 11-1. Therefore, the fluid sterilizing apparatus cannot appropriately control the emission of ultraviolet light from the first optoelectronic element upon receiving the first transmitted light reception signal alone, and thus there may be a case in which it is not possible to make constant the dose of ultraviolet light applied to the fluid F.

Here, the first light-transmitting member 40-1 and the light-transmitting member for transmitted light 70 are both connected to the chamber 30, and both contact the fluid F. Therefore, the types and thicknesses of contaminants adhering to the first light-transmitting member 40-1 and the light-transmitting member for transmitted light 70 are considered to be substantially the same. Therefore, the fluid sterilizing apparatus 100 can make constant the dose of ultraviolet light applied to the fluid F by controlling the emission of ultraviolet light from the first optoelectronic element 11-1 upon receiving the first reflected light reception signal, the first emitted light reception signal, and the first transmitted light reception signal.

The operations of the fluid sterilizing apparatus 100 for constantly irradiating the fluid F with a predetermined dose of ultraviolet light will be described in more detail.

First, the fluid sterilizing apparatus 100 supplies, to the first optoelectronic element 11-1, a drive current enough to apply a predetermined dose of ultraviolet light to the fluid F, from the power supply 51 of the control unit 50, in a state in which no contaminant adheres to the first light-transmitting member 40-1. The fluid sterilizing apparatus 100 stores, in the memory 52, the magnitude of the first reflected light reception signal obtained at this time as the first reflected light reception signal initial value. Also, the fluid sterilizing apparatus 100 stores, in the memory 52, the magnitude of the first emitted light reception signal obtained at this time as the first emitted light reception signal initial value. Further, the fluid sterilizing apparatus 100 stores, in the memory 52, the magnitude of the first transmitted light reception signal obtained at this time as the first transmitted light reception signal initial value.

As the operation of the fluid sterilizing apparatus 100 continues, contaminants adhere to the first light-transmitting member 40-1.

Here, there are a case in which the refractive index of the contaminants adhering to the first light-transmitting member 40-1 is higher than the refractive index of the first light-transmitting member 40-1, and a case in which the refractive index of the contaminants adhering to the first light-transmitting member 40-1 is lower than the refractive index of the first light-transmitting member 40-1.

When the refractive index of the contaminants adhering to the first light-transmitting member 40-1 is higher than the refractive index of the first light-transmitting member 40-1, even if the magnitude of the first emitted light reception signal is substantially the same as the first emitted light reception signal initial value, the magnitude of the first reflected light reception signal becomes larger than the first reflected light reception signal initial value. Moreover, the magnitude of the first transmitted light reception signal becomes smaller than the magnitude of the first transmitted light reception signal initial value. This is because the contaminants adhering to the first light-transmitting member 40-1 and the light-transmitting member for transmitted light 70 increase the reflectance of ultraviolet light at the interface between the first light-transmitting member 40-1 and the contaminants and at the interface between the light-transmitting member for transmitted light 70 and the contaminants, resulting in a decrease in the transmittance of the ultraviolet light.

On the other hand, when the refractive index of the contaminants adhering to the first light-transmitting member 40-1 is lower than the refractive index of the first light-transmitting member 40-1, even if the magnitude of the first emitted light reception signal is substantially the same as the first emitted light reception signal initial value, the magnitude of the first reflected light reception signal becomes smaller than the first reflected light reception signal initial value. Moreover, the magnitude of the first transmitted light reception signal becomes larger than the magnitude of the first transmitted light reception signal initial value. This is because the contaminants adhering to the first light-transmitting member 40-1 and the light-transmitting member for transmitted light 70 decrease the reflectance of ultraviolet light at the interface between the first light-transmitting member 40-1 and the contaminants and at the interface between the light-transmitting member for transmitted light 70 and the contaminants, resulting in an increase in the transmittance of the ultraviolet light.

Subsequently, the fluid sterilizing apparatus 100 supplies a drive current to the first optoelectronic element 11-1 from the power supply 51 of the control unit 50, and then causes the processor 53 to calculate the transmittance of the ultraviolet light at the interface between the first light-transmitting member 40-1 and the contaminants from the magnitudes of the first reflected light reception signal and the first emitted light reception signal. Also, the fluid sterilizing apparatus 100 causes the processor 53 to calculate the absorption rate of the ultraviolet light by the fluid F or the like from the magnitude of the first transmitted light reception signal. The fluid sterilizing apparatus 100 causes the processor 53 to adjust the drive current supplied to the first optoelectronic element 11-1 from the power supply 51 based on the calculation result of the transmittance of the ultraviolet light at the interface between the first light-transmitting member 40-1 and the contaminants and based on the calculation result of the absorption rate of the ultraviolet light by the fluid F or the like. Thus, the fluid sterilizing apparatus 100 can make constant the dose of ultraviolet light applied to the fluid F.

Subsequently, for making constant the dose of ultraviolet light applied to the fluid F, in accordance with an increase in the absorption of the ultraviolet light by the adhering and accumulating contaminants, the output of light from the first optoelectronic element 11-1 increases monotonically, and thus the magnitude of the first emitted light reception signal increases monotonically. The magnitude of the first reflected light reception signal increases or decreases from the start of operation of the fluid sterilizing apparatus 100, and then increases monotonically in proportion to the magnitude of the first emitted light reception signal. On the other hand, the magnitude of the first transmitted light reception signal decreases monotonically. When the absolute value of the difference between the magnitude of the first transmitted light reception signal and a magnitude of the first transmitted light reception signal initial value is significant and exceeds a predetermined transmitted light threshold, adhesion and accumulation of contaminants are significant, and the drive current of the first optoelectronic element 11-1 is increased. Continuing to operate the fluid sterilizing apparatus 100 in this state significantly reduces the lifetime of the first optoelectronic element 11-1. In this case, the control unit 50 causes the processor 53 to output a first error signal to the user interface unit 60.

As the operation of the fluid sterilizing apparatus 100 continues, the first optoelectronic element 11-1 is progressively degraded. This increases the drive current enough to irradiate the fluid F with a predetermined dose of ultraviolet light required for sterilization of the fluid F. When the magnitude of the drive current of the first optoelectronic element 11-1 exceeds the absolute maximum rating of the drive current of the first optoelectronic element 11-1, the first optoelectronic element 11-1 can break down suddenly. Therefore, there is a need to prevent sudden breakdown of the fluid sterilizing apparatus 100 by setting a predetermined current threshold for the drive current that is equal to or less than the absolute maximum rating of the drive current of the first optoelectronic element 11-1.

On the other hand, as described above, the drive current of the first optoelectronic element 11-1 increases in accordance with an increase in the absorption of ultraviolet light by the adhering and accumulating contaminants. In this case, the drive current of the first optoelectronic element 11-1 decreases after the removal of contaminants compared to before the removal of contaminants, replacement of the first optoelectronic element 11-1 is not needed. Therefore, when the absolute value of the difference between the magnitude of the first transmitted light reception signal and the magnitude of the first transmitted light reception signal initial value does not exceed a predetermined transmitted light threshold, and the magnitude of the drive current supplied to the first optoelectronic element 11-1 exceeds a predetermined current threshold, the control unit 50 causes the processor 53 to output a second error signal to the user interface unit 60 for preventing sudden breakdown of the fluid sterilizing apparatus 100.

As described above, the fluid sterilizing apparatus 100 can make constant the dose of ultraviolet light applied to the fluid F. Also, it is possible to improve maintainability by notifying an appropriate timing of maintenance of the fluid sterilizing apparatus 100, and prevent sudden breakdown of the fluid sterilizing apparatus 100.

In the first embodiment of the present disclosure, the memory 52 can store at least the first transmitted light reception signal initial value. The control unit 50 can output a first error signal when the absolute value of the difference between the magnitude of the first transmitted light reception signal and the magnitude of the first transmitted light reception signal initial value exceeds a predetermined transmitted light threshold. The user interface unit 60 receives an input of the first error signal, and then lights the indicator lamp 62 and sounds the alarm 63 for notification of the state in which contaminants are adhering to the first light-transmitting member 40-1. Alternatively, the user interface unit 60 causes the display 61 to display a text message notifying the state in which contaminants are adhering to the first light-transmitting member 40-1. This can notify the operator of the fluid sterilizing apparatus 100 of the state in which contaminants are adhering to the first light-transmitting member 40-1, and recommend cleaning of the first light-transmitting member 40-1. By recommending the cleaning at an appropriate timing, it is possible to improve maintainability of the fluid sterilizing apparatus 100.

The memory 52 stores the first transmitted light reception signal initial value, the predetermined current threshold for the drive current, and the predetermined transmitted light threshold. When the absolute value of the difference between the magnitude of the first transmitted light reception signal and the magnitude of the first transmitted light reception signal initial value does not exceed the transmitted light threshold, and the magnitude of the drive current supplied to the first optoelectronic element 11-1 exceeds the current threshold, the control unit 50 can output a second error signal indicating degradation in the first optoelectronic element 11-1. The user interface unit 60 receives the second error signal, and then lights the indicator lamp 62 and sounds the alarm 63 for notification of the state in which the first optoelectronic element 11-1 is degraded. Alternatively, the user interface unit 60 causes the display 61 to display a text message notifying the state in which the first optoelectronic element 11-1 is degraded. This can notify the operator of the fluid sterilizing apparatus 100 of the state in which the first optoelectronic element 11-1 is degraded, and recommend replacing the first optoelectronic element 11-1. By recommending the replacement at an appropriate timing, it is possible to improve maintainability of the fluid sterilizing apparatus 100.

The first optoelectronic element for emitted light 12-1 can receive ultraviolet light emitted from the first optoelectronic element 11-1 and reflected by the first cover 152-1. By this, the first optoelectronic element for emitted light 12-1 can output, to the control unit 50, the first emitted light reception signal related to the output of the ultraviolet light emitted from the first optoelectronic element 11-1 without receiving any impact of the contaminants on the first light-transmitting member 40-1 or the like. The fluid sterilizing apparatus 100 can obtain information of the lifetime of the first optoelectronic element 11-1 based on the first emitted light reception signal.

The first cover 152-1 includes a first light quantity adjusting member 153-1, and the first light quantity adjusting member 153-1 can be disposed to face the first optoelectronic element for emitted light 12-1. The first light quantity adjusting member 153-1 can reduce light incident on the first optoelectronic element for emitted light 12-1 from the exterior of the first housing 151-1, and can reduce noise of the first emitted light reception signal. Thus, the fluid sterilizing apparatus 100 can improve the control accuracy of the output of the ultraviolet light emitted from the first optoelectronic element 11-1 based on the first emitted light reception signal.

The fluid sterilizing apparatus 100 can include the first housing 151-1 that houses the first optoelectronic element 11-1 and the first optoelectronic element for emitted light 12-1, and supports the first cover 152-1. This configuration enhances the airtightness of the compartment housing the first optoelectronic element 11-1 and the first optoelectronic element for emitted light 12-1, thereby mitigating degradation in the first optoelectronic element 11-1 and the first optoelectronic element for emitted light 12-1.

The first optoelectronic element 11-1 and the first optoelectronic element for reflected light 13-1 can be disposed to face the first light-transmitting member 40-1. Thus, the first optoelectronic element for reflected light 13-1 can receive the ultraviolet light emitted from the first optoelectronic element 11-1 and reflected at the interface between the first light-transmitting member 40-1 and the fluid F, and can output the first reflected light reception signal to the control unit 50. The fluid sterilizing apparatus 100 can obtain information of the contaminants on the first light-transmitting member 40-1 based on the first reflected light reception signal or the like.

In the first embodiment of the present disclosure, the first light source 10-1 (light source) includes the first optoelectronic element 11-1 (first light-emitting element), and the first optoelectronic element for emitted light 12-1 (first light-receiving element for emitted light) configured to receive a part of the ultraviolet light (light) from the first optoelectronic element 11-1. Also, the first light source 10-1 includes the first light-transmitting member 40-1 configured to reflect a part of the ultraviolet light from the first optoelectronic element 11-1, and the first optoelectronic element for reflected light 13-1 (first light-receiving element for reflected light) configured to receive a part of the ultraviolet light reflected by the first light-transmitting member 40-1.

The first light source 10-1 may further include the first cover 152-1. The first optoelectronic element for emitted light 12-1 can receive the ultraviolet light emitted from the first optoelectronic element 11-1 and reflected by the first cover 152-1. The first cover 152-1 includes the first light quantity adjusting member 153-1. The first light quantity adjusting member 153-1 can be disposed to face the first optoelectronic element for emitted light 12-1.

The first light source 10-1 can control the emission of the ultraviolet light from the first optoelectronic element 11-1 by mitigating the impact of the contaminants adhering to the first light-transmitting member 40-1 based on a light reception signal of the first optoelectronic element for emitted light 12-1 and a light reception signal of the first optoelectronic element for reflected light 13-1. For example, the first light source 10-1 can control the emission of the ultraviolet light from the first optoelectronic element 11-1 by adjusting the drive current supplied to the first optoelectronic element 11-1, thereby adjusting the dose of the ultraviolet light within a predetermined range. The first light source 10-1 can make constant the dose of the ultraviolet light to be applied by controlling the emission of the ultraviolet light from the first optoelectronic element 11-1 while mitigating the impact of the contaminants adhering to the first light-transmitting member 40-1.

In the first embodiment of the present disclosure, a light source apparatus includes: the first optoelectronic element 11-1, the first optoelectronic element for emitted light 12-1, and the first optoelectronic element for reflected light 13-1; and the first light-transmitting member 40-1 disposed to face the first optoelectronic element 11-1, the first optoelectronic element for emitted light 12-1, and the first optoelectronic element for reflected light 13-1. Also, the light source apparatus includes the control unit 50 including the processor 53 configured to control the emission of light from the first optoelectronic element 11-1 upon receiving the first reflected light reception signal generated from the first optoelectronic element for reflected light 13-1 upon receiving light (ultraviolet light) emitted from the first optoelectronic element 11-1 and reflected by the first light-transmitting member 40-1, and the first emitted light reception signal generated from the first optoelectronic element for emitted light 12-1 upon receiving a part of the ultraviolet light emitted from the first optoelectronic element 11-1.

The light source apparatus can control, based on the first emitted light reception signal and the first reflected light reception signal, the emission of the ultraviolet light from the first optoelectronic element 11-1 while mitigating the impact of the contaminants adhering to the first light-transmitting member 40-1. For example, by adjusting the drive current supplied to the first optoelectronic element 11-1, the light source apparatus can control the ultraviolet light emitted from the first optoelectronic element 11-1, and adjust the dose of ultraviolet light within a predetermined range. The first light source 10-1 can make constant the dose of ultraviolet light by controlling the emission of the ultraviolet light from the first optoelectronic element 11-1 while mitigating the impact of the contaminants adhering to the first light-transmitting member 40-1.

From the viewpoint of extending the lifetime of the first optoelectronic element 11-1, it is preferable to cause the fluid sterilizing apparatus 100 to emit ultraviolet light in the above-described manner. Specifically, first, in the initial stage in which use of the fluid sterilizing apparatus 100 begins, a drive current is supplied to the first optoelectronic element 11-1 for causing the fluid sterilizing apparatus 100 to emit a predetermined dose of ultraviolet light required for sterilization of the fluid F. Subsequently, as the period of use of the fluid sterilizing apparatus 100 becomes longer, the drive current supplied to the first optoelectronic element 11-1 is gradually increased for causing the fluid sterilizing apparatus 100 to emit the predetermined dose of ultraviolet light.

### Second Embodiment

Next, a fluid sterilizing apparatus according to a second embodiment of the present disclosure will be described. Note that names and symbols the same as those in the already described embodiment indicate the same or similar members or configurations, and detailed description thereof will be appropriately omitted. The same applies to a third embodiment described below.

<Configuration of Fluid Sterilizing Apparatus according to Second Embodiment of Present Disclosure>

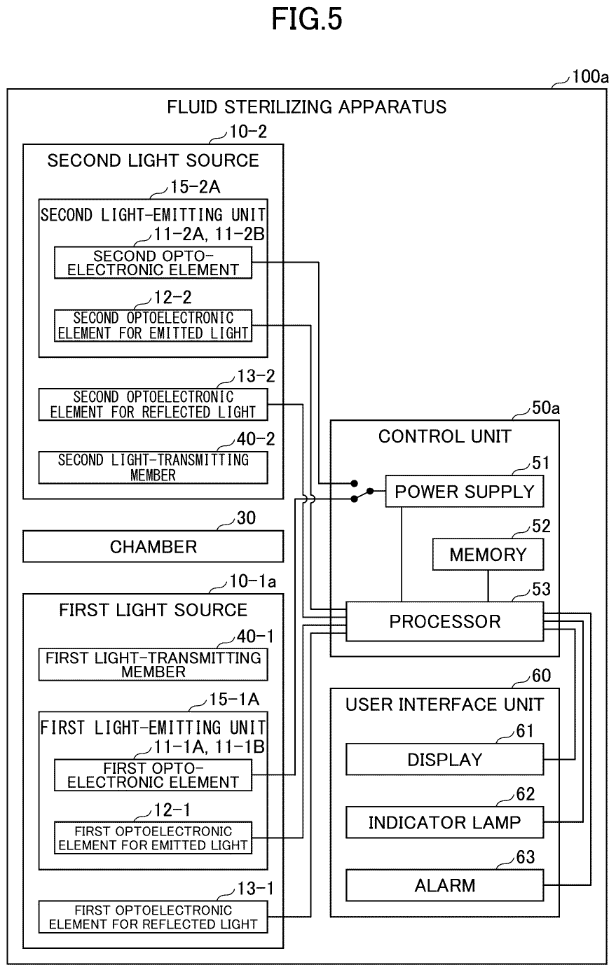

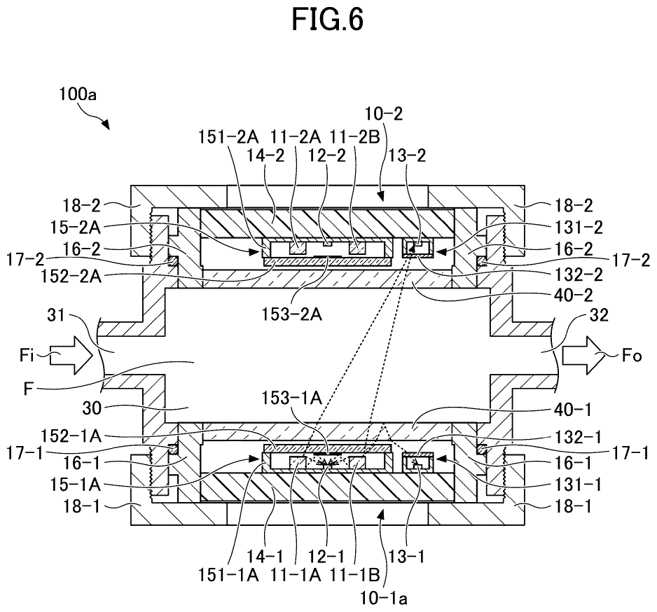

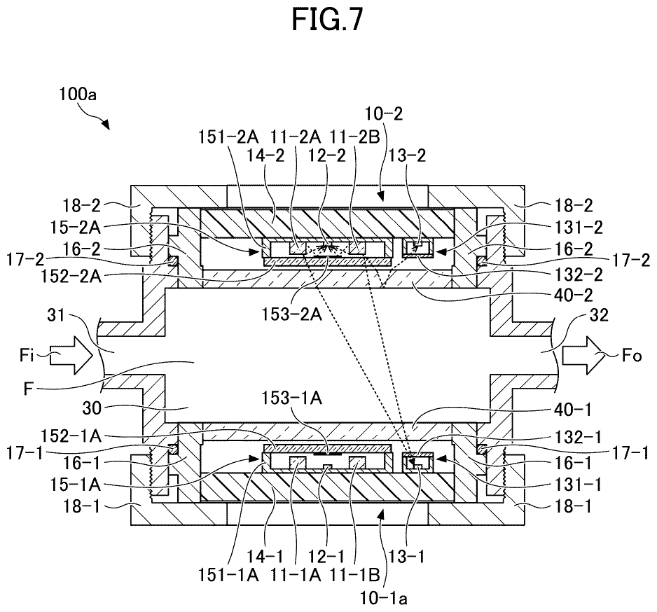

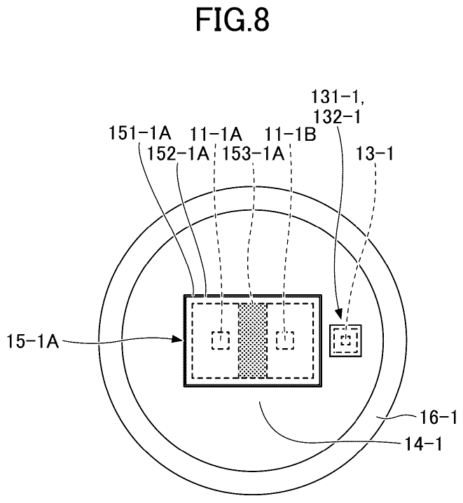

The configuration of the fluid sterilizing apparatus according to the second embodiment of the present disclosure will be described with reference to FIGS. 5 to 8. FIG. 5 is a block diagram illustrating the overall configuration of a fluid sterilizing apparatus 100a according to the second embodiment of the present disclosure. FIG. 6 is a schematic cross-sectional view illustrating a state I of the fluid sterilizing apparatus 100a. FIG. 7 is a schematic cross-sectional view illustrating a state II of the fluid sterilizing apparatus 100a. FIG. 8 is a schematic top view illustrating the configuration around a first optoelectronic element 11-1A and a first optoelectronic element 11-1B included in the fluid sterilizing apparatus 100a.

### (Overall Configuration)

As illustrated in FIGS. 5 to 7, the fluid sterilizing apparatus 100a includes a first light source 10-1a, the chamber 30, a second light source 10-2, and a control unit 50a. The first light source 10-1a includes the first light-transmitting member 40-1. The second light source 10-2 includes a second light-transmitting member 40-2. FIGS. 6 and 7 illustrate cross sections of the fluid sterilizing apparatus 100a including the first light source 10-1a, the chamber 30, and the second light source 10-2.

The fluid sterilizing apparatus 100a may include one or more first optoelectronic elements, and may include one or more second optoelectronic elements. Also, the fluid sterilizing apparatus 100a may include one or more first optoelectronic elements for emitted light, and may include one or more second optoelectronic elements for emitted light. The same configuration of the first optoelectronic element 11-1 can be used as the configuration of the one or more first optoelectronic elements and the one or more second optoelectronic elements. The same configuration of the first optoelectronic element for emitted light 12-1 can be used as the configuration of the one or more first optoelectronic elements for emitted light and the one or more second optoelectronic elements for emitted light.

The first light source 10-1a includes a first optoelectronic element 11-1A, a first optoelectronic element 11-1B, the first optoelectronic element for emitted light 12-1, and the first optoelectronic element for reflected light 13-1. The second light source 10-2 includes a second optoelectronic element 11-2A, a second optoelectronic element 11-2B, a second optoelectronic element for emitted light 12-2, and a second optoelectronic element for reflected light 13-2. The first light source 10-1 a and the second light source 10-2 are disposed to face each other.

In the fluid sterilizing apparatus 100a, the control unit 50a includes the processor 53 configured to alternately switch between a state I in which the first light source 10-1a emits ultraviolet light and the second light source 10-2 does not emit ultraviolet light, and a state II in which the second light source 10-2 emits ultraviolet light and the first light source 10-1a does not emit ultraviolet light. FIG. 5 illustrates a case in which the control unit 50a is in the state I. In the state I, the second optoelectronic element for reflected light 13-2 functions as an optoelectronic element for transmitted light, and emits the first transmitted light reception signal. The control unit 50a controls the emission of ultraviolet light from the first optoelectronic element 11-1A and the first optoelectronic element 11-1B based on the first reflected light reception signal, the first emitted light reception signal, and the first transmitted light reception signal. In the state II, the first optoelectronic element for reflected light 13-1 functions as the optoelectronic element for transmitted light, and emits a second transmitted light reception signal. The control unit 50a controls the emission of ultraviolet light from the second optoelectronic element 11-2A and the second optoelectronic element 11-2B based on the second reflected light reception signal, the second emitted light reception signal, and the second transmitted light reception signal.

The above is a main difference of the fluid sterilizing apparatus 100a from the fluid sterilizing apparatus 100 according to the first embodiment of the present disclosure.

Here, the junction temperature of the first or second optoelectronic element increases when the first or second optoelectronic element starts emitting ultraviolet light, and decreases when the first or second optoelectronic element stops emitting the ultraviolet light and returns to the temperature before the first or second optoelectronic element emits ultraviolet light. The fluid sterilizing apparatus 100a emits ultraviolet light alternately from the first light source 10-1a and from the second light source 10-2, and does not emit ultraviolet light from any light source other than the light source emitting the ultraviolet light. Thus, the fluid sterilizing apparatus 100a can reduce an increase in the junction temperature of the first optoelectronic element 11-1A, the first optoelectronic element 11-1B, the second optoelectronic element 11-2A, and the second optoelectronic element 11-2B. This extends the lifetime of the first optoelectronic element 11-1A, the first optoelectronic element 11-1B, the second optoelectronic element 11-2A, or the second optoelectronic element 11-2B, and thus extends a cycle of replacement of the first optoelectronic element 11-1A, the first optoelectronic element 11-1B, the second optoelectronic element 11-2A, or the second optoelectronic element 11-2B.

Because the cycle of replacement of the first optoelectronic element 11-1A, the first optoelectronic element 11-1B, the second optoelectronic element 11-2A, or the second optoelectronic element 11-2B is extended, it is possible to improve maintainability of the fluid sterilizing apparatus 100a in the second embodiment of the present disclosure. Also, by causing the first optoelectronic element for reflected light 13-1 and the second optoelectronic element for reflected light 13-2, not functioning as an optoelectronic element for reflected light, to function as an optoelectronic element for transmitted light, it is possible to reduce the number of parts of the fluid sterilizing apparatus 100a, thereby enabling achieving a simplified configuration and a reduced cost in the second embodiment of the present disclosure.

Hereinafter, the fluid sterilizing apparatus 100a will be described in more detail.

### (First Light Source 10-1a)

In the example illustrated in FIGS. 6 and 7, the first light source 10-1a includes the first optoelectronic element 11-1A, the first optoelectronic element 11-1B, the first optoelectronic element for emitted light 12-1, and the first optoelectronic element for reflected light 13-1. The first light source 10-1a includes a first light-emitting unit 15-1A disposed at the first substrate 14-1, a first housing for reflected light 131-1, and a first cover for reflected light 132-1.

The first optoelectronic element 11-1A and the first optoelectronic element 11-1B are connected in parallel.

The first light-emitting unit 15-1A includes a first housing 151-1A and a first cover 152-1A. The first housing 151-1A houses the first optoelectronic element 11-1A, the first optoelectronic element 11-1B, and the first optoelectronic element for emitted light 12-1, and supports the first cover 152-1A.

The first cover 152-1A is disposed to face the first optoelectronic element 11-1A, the first optoelectronic element 11-1B, and the first optoelectronic element for emitted light 12-1 and disposed between: the first optoelectronic element 11-1A, the first optoelectronic element 11-1B, and the first optoelectronic element for emitted light 12-1; and the first light-transmitting member 40-1. The first cover 152-1A transmits a part of the ultraviolet light emitted from the first optoelectronic element 11-1A. The first cover 152-1A includes a first light quantity adjusting member 153-1A. The first light quantity adjusting member 153-1A is disposed to face the first optoelectronic element for emitted light 12-1.

The first light-emitting unit 15-1A and a second light-emitting unit 15-2A can be configured in the same manner as in the first light-emitting unit 15-1.

The first housing for reflected light 131-1 is a member that houses the first optoelectronic element for reflected light 13-1, and supports the first cover for reflected light 132-1. The first cover for reflected light 132-1 is disposed to face the first optoelectronic element for reflected light 13-1 and disposed between the first optoelectronic element for reflected light 13-1 and the first light-transmitting member 40-1. The first cover for reflected light 132-1 transmits a part of the ultraviolet light emitted from the first optoelectronic element 11-1A, the first optoelectronic element 11-1B, the second optoelectronic element 11-2A, or the second optoelectronic element 11-2B. The first housing for reflected light 131-1 can be configured in the same manner as in the housing for transmitted light 211. The first cover for reflected light 132-1 can be configured in the same manner as in the cover for transmitted light 212.

In the example illustrated in FIG. 8, the first substrate 14-1 has an outer peripheral shape that is substantially circular in a top view. The first frame 16-1 has a hollow circular shape in a top view, and the first substrate 14-1 is disposed in the inner space of the first frame 16-1. However, the first substrate 14-1 may have an outer peripheral shape that is substantially rectangular, substantially elliptical, substantially polygonal, or the like in a top view. The first frame 16-1 may have a hollow rectangular shape in a top view, and the first substrate 14-1 may be disposed in the inner space of the first frame 16-1.

The first housing 151-1A and the first cover 152-1A hermetically seal the first optoelectronic element 11-1A, the first optoelectronic element 11-1B, and the first optoelectronic element for emitted light 12-1 from the exterior of the first light-emitting unit 15-1A. The first housing 151-1A can be configured in the same manner as in the first housing 151-1. The first cover 152-1A can be configured in the same manner as in the first cover 152-1. In the example illustrated in FIG. 8, the first housing 151-1A and the first cover 152-1A have an outer peripheral shape that is substantially rectangular in a top view. However, the first housing 151-1A and the first cover 152-1A may have an outer peripheral shape that is substantially circular, substantially elliptical, or substantially polygonal in a top view.

### (First Light-Transmitting Member 40-1)

The first light-transmitting member 40-1 is disposed between: the first optoelectronic element 11-1A, the first optoelectronic element 11-1B, the first optoelectronic element for emitted light 12-1, and the first optoelectronic element for reflected light 13-1; and the inner space of the chamber 30.

Similar to the first light source 10-1, the first light source 10-1a can be removed from the fluid sterilizing apparatus 100a. The ability to remove the first light source 10-1a from the fluid sterilizing apparatus 100a facilitates removal of the contaminants adhering to the first light-transmitting member 40-1, replacement of the first substrate 14-1 over which the first light-emitting unit 15-1A and the first housing for reflected light 131-1 are placed, or the like.

### (Second Light Source 10-2)

In the example illustrated in FIGS. 6 and 7, the second light source 10-2 includes the second optoelectronic element 11-2A, the second optoelectronic element 11-2B, the second optoelectronic element for emitted light 12-2, and the second optoelectronic element for reflected light 13-2. Also, the second light source 10-2 includes a second substrate 14-2, the second light-emitting unit 15-2A disposed below the second substrate 14-2, a second housing for reflected light 131-2, and a second cover for reflected light 132-2. Further, the second light source 10-2 includes a second frame 16-2 that supports the second substrate 14-2 and the second light-transmitting member 40-2, a second sealing member 17-2 disposed between the chamber 30 and the second frame 16-2, and a second fixing member 18-2 that fixes the second light source 10-2 to the chamber 30.

The second optoelectronic element 11-2A and the second optoelectronic element 11-2B are connected in parallel.

The second optoelectronic element 11-2A, the second optoelectronic element 11-2B, the second optoelectronic element for emitted light 12-2, and the second optoelectronic element for reflected light 13-2 are disposed to face the first optoelectronic element 11-1A, the first optoelectronic element 11-1B, the first optoelectronic element for emitted light 12-1, and the first optoelectronic element for reflected light 13-1.

The second light-emitting unit 15-2A includes a second housing 151-2A and a second cover 152-2A. The second housing 151-2A is a member that houses the second optoelectronic element 11-2A, the second optoelectronic element 11-2B, and the second optoelectronic element for emitted light 12-2, and supports the second cover 152-2A. The second cover 152-2A is disposed to face the second optoelectronic element 11-2A and the second optoelectronic element for emitted light 12-2 and disposed between: the second optoelectronic element 11-2A, the second optoelectronic element 11-2B, and the second optoelectronic element for emitted light 12-2; and the second light-transmitting member 40-2. The second cover 152-2A transmits a part of the ultraviolet light emitted from the second optoelectronic element 11-2A. The second cover 152-2A includes a second light quantity adjusting member 153-2A. The second light quantity adjusting member 153-2A is disposed to face the second optoelectronic element for emitted light 12-2.

A second housing for reflected light 131-2 is a member that houses the second optoelectronic element for reflected light 13-2, and supports the second cover for reflected light 132-2. The second cover for reflected light 132-2 is disposed to face the second optoelectronic element for reflected light 13-2, and disposed between the second optoelectronic element for reflected light 13-2 and the second light-transmitting member 40-2. The second cover for reflected light 132-2 transmits a part of the ultraviolet light emitted from the first optoelectronic element 11-1A, the first optoelectronic element 11-1B, the second optoelectronic element 11-2A, or the second optoelectronic element 11-2B.

The second substrate 14-2 can be configured in the same manner as in the first substrate 14-1. The second frame 16-2 can be configured in the same manner as in the first frame 16-1. The second housing 151-2A can be configured in the same manner as in the first housing 151-1A. The second cover 152-2A can be configured in the same manner as in the first cover 152-1A.

### (Second Light-Transmitting Member 40-2)

The second light-transmitting member 40-2 is disposed between: the second optoelectronic element 11-2A, the second optoelectronic element 11-2B, and the second optoelectronic element for emitted light 12-2, and the second optoelectronic element for reflected light 13-2; and the inner space of the chamber 30.

The second light source 10-2 can be removed from the fluid sterilizing apparatus 100a. The ability to remove the second light source 10-2 from the fluid sterilizing apparatus 100a facilitates removal of the contaminants adhering to the second light-transmitting member 40-2, replacement of the second substrate 14-2 over which the second light-emitting unit 15-2A and the second housing for reflected light 131-2 are placed, or the like. The second sealing member 17-2 ensures water tightness between the second light source 10-2 and the inner space of the chamber 30.

An internal thread is formed at the inner surface of the second fixing member 18-2. The internal thread of the second fixing member 18-2 is screw-connected with an external thread formed at a portion of the chamber 30 corresponding to the internal thread. By screwing and tightening the second fixing member 18-2 and pressing the second frame 16-2 against the chamber 30, the second light source 10-2 can be attached and fixed to the chamber 30. The second fixing member 18-2 can be removed from the chamber 30 by loosening the screw connection between the internal thread of the second fixing member 18-2 and the external thread of the chamber 30. By removing the second fixing member 18-2 from the chamber 30, the second light source 10-2 can be removed from the chamber 30.

### (Chamber 30)

The fluid F located in the inner space of the chamber 30 is disposed between: the first optoelectronic element 11-1A, the first optoelectronic element 11-1B, the first optoelectronic element for emitted light 12-1, and the first optoelectronic element for reflected light 13-1; and the second optoelectronic element 11-2A, the second optoelectronic element 11-2B, the second optoelectronic element for emitted light 12-2, and the second optoelectronic element for reflected light 13-2. The fluid F located in the inner space of the chamber 30 is irradiated with the ultraviolet light emitted from the first optoelectronic element 11-1A and the first optoelectronic element 11-1B, or the second optoelectronic element 11-2A and the second optoelectronic element 11-2B.

### (Control Unit 50a)

The control unit 50a includes the processor 53 configured to alternately switch between the state I and the state II. The processor 53 included in the control unit 50a can alternately switch between the state I and the state II by alternately switching the drive current supplied to the first light source 10-1a (the first optoelectronic element 11-1A and the first optoelectronic element 11-1B) or the second light source 10-2 (the second optoelectronic element 11-2A and the second optoelectronic element 11-2B).

### (State I)

The state I is a state in which the first optoelectronic element 11-1A and the first optoelectronic element 11-1B emit ultraviolet light, and the first optoelectronic element for emitted light 12-1, the first optoelectronic element for reflected light 13-1, the second optoelectronic element 11-2A, the second optoelectronic element 11-2B, the second optoelectronic element for emitted light 12-2, and the second optoelectronic element for reflected light 13-2 do not emit ultraviolet light.

In the example illustrated in FIGS. 5 and 6, in the state I, the first optoelectronic element for reflected light 13-1 outputs the first reflected light reception signal upon receiving the ultraviolet light emitted from the first optoelectronic element 11-1A and the first optoelectronic element 11-1B and reflected by the first light-transmitting member 40-1. Also, the first optoelectronic element for emitted light 12-1 outputs the first emitted light reception signal upon receiving a part of the ultraviolet light emitted from the first optoelectronic element 11-1A and the first optoelectronic element 11-1B. Further, the second optoelectronic element for reflected light 13-2 outputs the first transmitted light reception signal upon receiving the ultraviolet light emitted from the first optoelectronic element 11-1A and the first optoelectronic element 11-1B and subsequently transmitted through the first light-transmitting member 40-1, the fluid F, and the second light-transmitting member 40-2. In addition, the control unit 50a controls the emission of the ultraviolet light from the first optoelectronic element 11-1A and the first optoelectronic element 11-1B based on the first reflected light reception signal, the first emitted light reception signal, and the first transmitted light reception signal.

For example, the control unit 50a causes the processor 53 to receive the first reflected light reception signal, the first emitted light reception signal, and the first transmitted light reception signal, execute signal processing of the received signals, and adjust the drive currents of the first optoelectronic element 11-1A and the first optoelectronic element 11-1B. This can control the emission of the ultraviolet light from the first optoelectronic element 11-1A and the first optoelectronic element 11-1B.

The first emitted light reception signal includes information of outputs of light emitted by the first optoelectronic element 11-1A and the first optoelectronic element 11-1B. The first emitted light reception signal is not impacted by the second light-transmitting member 40-2, the contaminants adhering to the second light-transmitting member 40-2, the fluid F, or the like. The first reflected light reception signal accounts for effects, such as light absorption, scattering, or the like, caused by at least one of the first cover 152-1A, the first light-transmitting member 40-1, or the contaminants adhering to the first light-transmitting member 40-1. The first transmitted light reception signal accounts for effects, such as light absorption, scattering, or the like, caused by at least one of the first cover 152-1A, the first light-transmitting member 40-1, the contaminants adhering to the first light-transmitting member 40-1, the fluid F, the contaminants adhering to the second light-transmitting member 40-2, or the second light-transmitting member 40-2.

### (State II)

The state II is a state in which the second optoelectronic element 11-2A and the second optoelectronic element 11-2B emit ultraviolet light, and the first optoelectronic element 11-1A, the first optoelectronic element 11-1B, the first optoelectronic element for emitted light 12-1, the first optoelectronic element for reflected light 13-1, the second optoelectronic element for emitted light 12-2, and the second optoelectronic element for reflected light 13-2 do not emit ultraviolet light.

In the example illustrated in FIG. 7, in the state II, the second optoelectronic element for reflected light 13-2 outputs the second reflected light reception signal upon receiving the ultraviolet light emitted from the second optoelectronic element 11-2A and the second optoelectronic element 11-2B and reflected by the second light-transmitting member 40-2. Also, the second optoelectronic element for emitted light 12-2 outputs the second emitted light reception signal upon receiving a part of the ultraviolet light emitted from the second optoelectronic element 11-2A and the second optoelectronic element 11-2B. Further, the first optoelectronic element for reflected light 13-1 outputs the second transmitted light reception signal upon receiving the ultraviolet light emitted from the second optoelectronic element 11-2A and the second optoelectronic element 11-2B and subsequently transmitted through the second light-transmitting member 40-2, the fluid F, and the first light-transmitting member 40-1. In addition, the control unit 50a controls the emission of the ultraviolet light from the second optoelectronic element 11-2A and the second optoelectronic element 11-2B based on the second reflected light reception signal, the second emitted light reception signal, and the second transmitted light reception signal.

For example, the control unit 50a causes the processor 53 to receive the second reflected light reception signal, the second emitted light reception signal, and the second transmitted light reception signal, execute signal processing of the received signals, and adjust the drive currents of the second optoelectronic element 11-2A and the second optoelectronic element 11-2B. This can control the emission of the ultraviolet light from the second optoelectronic element 11-2A and the second optoelectronic element 11-2B.

The second emitted light reception signal includes information of outputs of light emitted from the second optoelectronic element 11-2A and the second optoelectronic element 11-2B. The second emitted light reception signal is not impacted by the first light-transmitting member 40-1, the contaminants adhering to the first light-transmitting member 40-1, the fluid F, or the like. The second reflected light reception signal accounts for effects, such as light absorption, scattering, or the like, caused by at least one of the second cover 152-2A, the second light-transmitting member 40-2, or the contaminants adhering to the second light-transmitting member 40-2. The second transmitted light reception signal accounts for effects, such as light absorption, scattering, or the like, caused by at least one of the second cover 152-2A, the first light-transmitting member 40-1, the contaminants adhering to the first light-transmitting member 40-1, the fluid F, the contaminants adhering to the second light-transmitting member 40-2, or the second light-transmitting member 40-2.

In the second embodiment of the present disclosure, the memory 52 can store at least the first transmitted light reception signal initial value and the second transmitted light reception signal initial value. The fluid sterilizing apparatus 100a supplies, to the first optoelectronic elements 11-1A and 11-1B, a drive current enough to apply a predetermined dose of ultraviolet light to the fluid F, from the power supply 51 of the control unit 50, in a state in which no contaminant adheres to the first light-transmitting member 40-1 and the second light-transmitting member 40-2. The fluid sterilizing apparatus 100a stores, in the memory 52, the magnitude of the first transmitted light reception signal obtained at this time as the first transmitted light reception signal initial value. Also, in this state, the fluid sterilizing apparatus 100a supplies, to the second optoelectronic elements 11-2A and 11-2B, a drive current enough to apply a predetermined dose of ultraviolet light to the fluid F from the power supply 51, and stores, in the memory 52, the magnitude of the second transmitted light reception signal obtained at this time as the second transmitted light reception signal initial value.

In the state I, the operation of the fluid sterilizing apparatus 100a when the control unit 50a outputs the first error signal and the second error signal is the same as the operation of the fluid sterilizing apparatus 100 in the first embodiment. In the state II, the operation of the fluid sterilizing apparatus 100a when the control unit 50a outputs the first error signal and the second error signal is the same as the operation of the fluid sterilizing apparatus 100 in the first embodiment except that the first emitted light reception signal is replaced with the second emitted light reception signal, the first reflected light reception signal is replaced with the second reflected light reception signal, and the first transmitted light reception signal is replaced with the second transmitted light reception signal.

<Effects of Fluid Sterilizing Apparatus 100a>

Next, effects of the fluid sterilizing apparatus 100a other than those described above will be described.

In the second embodiment of the present disclosure, the first optoelectronic element 11-1A and the first optoelectronic element 11-1B are connected in parallel, and the second optoelectronic element 11-2A and the second optoelectronic element 11-2B are connected in parallel. This arrangement can drive, in parallel, a plurality of the first optoelectronic elements and a plurality of the second optoelectronic elements. Also, it is possible to readily control the emissions of ultraviolet light from the plurality of the first optoelectronic elements and the plurality of the second optoelectronic elements. Further, when the plurality of the first optoelectronic elements and the plurality of the second optoelectronic elements emit ultraviolet light, it is possible to increase the dose of ultraviolet light emitted from the first light source 10-1a and the second light source 10-2 in the fluid sterilizing apparatus 100a.

The effects other than those described above in the fluid sterilizing apparatus 100a according to the second embodiment of the present disclosure are the same as the effects of the fluid sterilizing apparatus 100 described in the first embodiment of the present disclosure.

### Third Embodiment

Next, a fluid sterilizing apparatus according to a third embodiment of the present disclosure will be described.

<Configuration of Fluid Sterilizing Apparatus according to Third Embodiment of Present Disclosure>

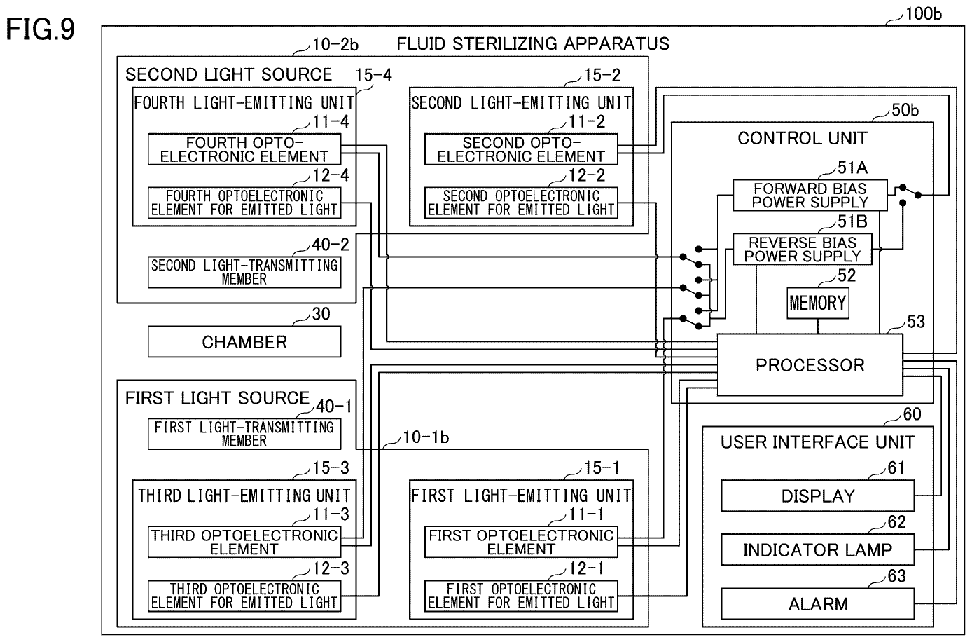

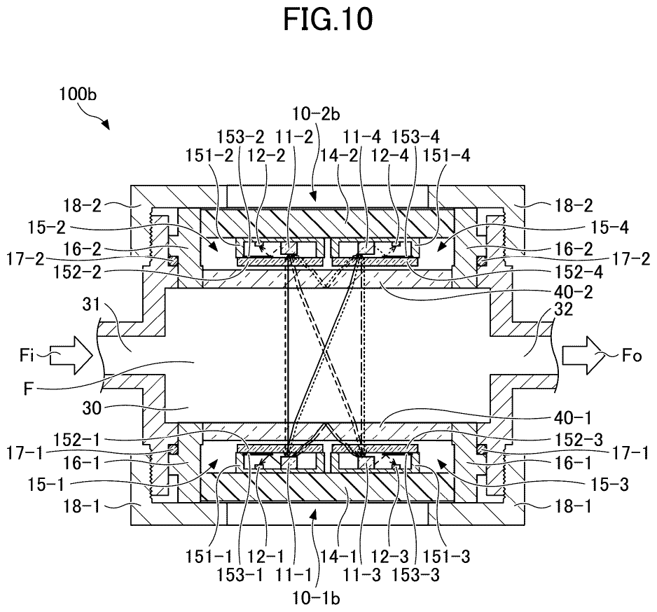

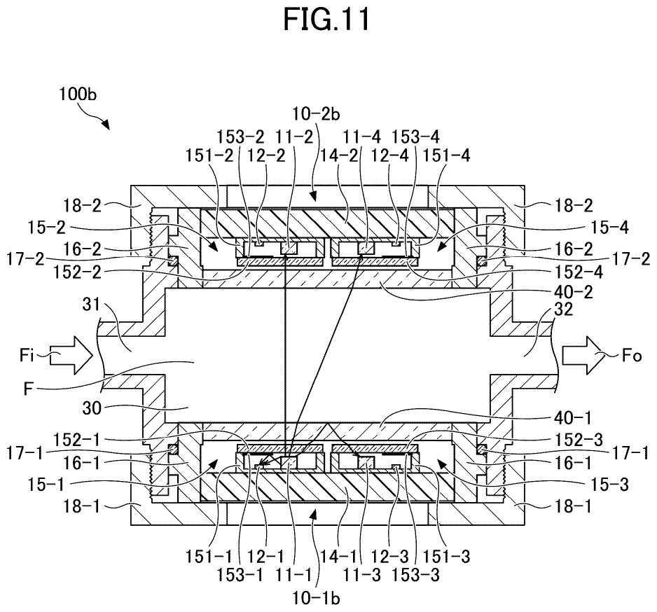

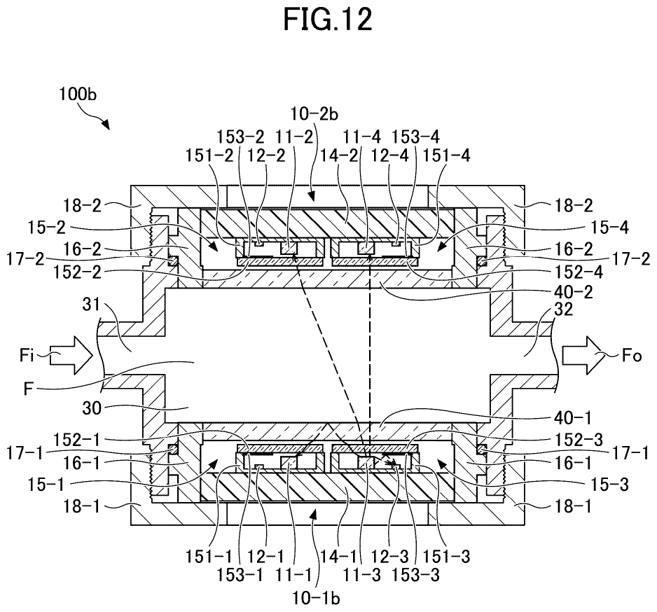

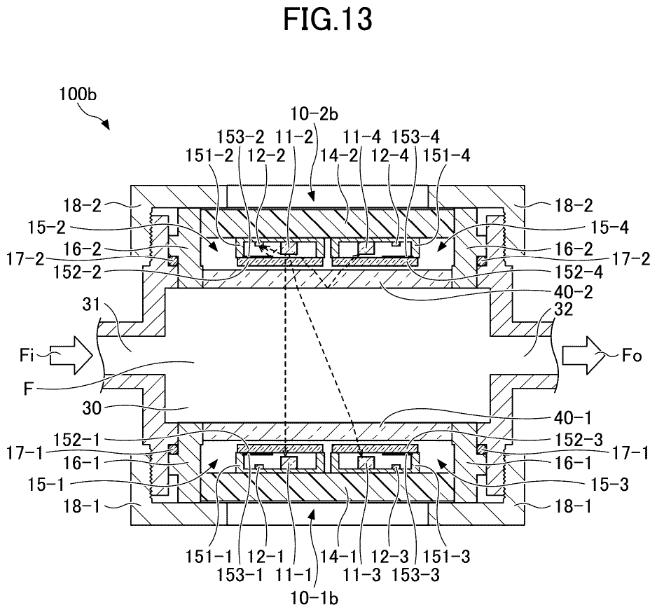

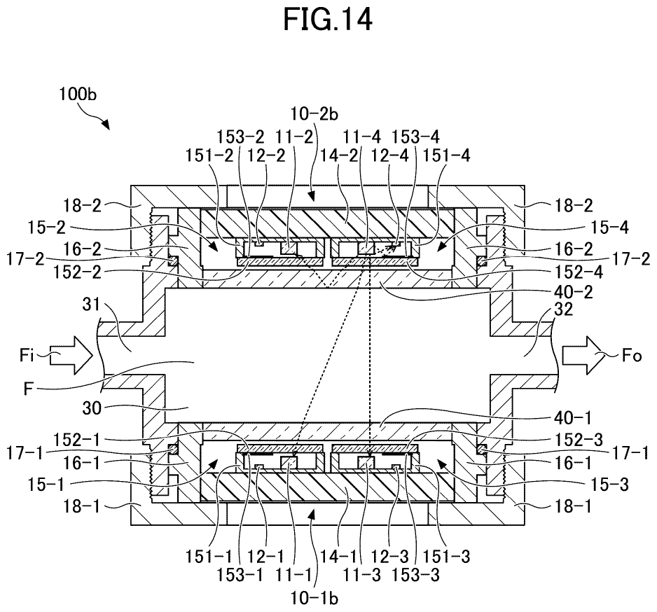

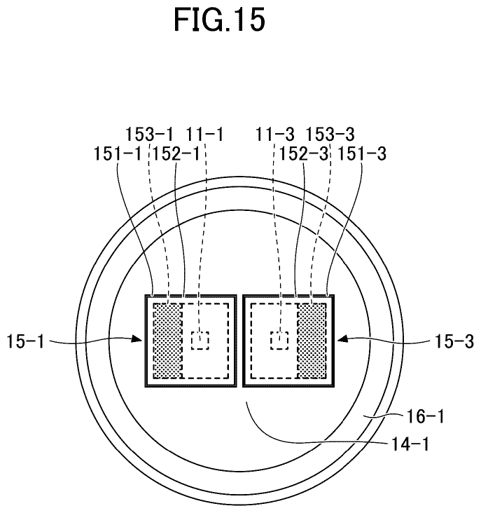

The configuration of the fluid sterilizing apparatus according to the third embodiment of the present disclosure will be described with reference to FIGS. 9 to 15. FIG. 9 is a block diagram illustrating an overall configuration of a fluid sterilizing apparatus 100b according to the third embodiment of the present disclosure. FIG. 10 is a schematic cross-sectional view illustrating a configuration of the fluid sterilizing apparatus in the vicinity of the chamber 30 included in the fluid sterilizing apparatus 100b. FIG. 11 is a schematic cross-sectional view illustrating a first state of the fluid sterilizing apparatus 100b. FIG. 12 is a schematic cross-sectional view illustrating a second state of the fluid sterilizing apparatus 100b. FIG. 13 is a schematic cross-sectional view illustrating a third state of the fluid sterilizing apparatus 100b. FIG. 14 is a schematic cross-sectional view illustrating a fourth state of the fluid sterilizing apparatus 100b. FIG. 15 is a schematic top view illustrating a configuration around the first optoelectronic element 11-1 and a third optoelectronic element 11-3 included in the fluid sterilizing apparatus 100b.

### (Overall Configuration)

As illustrated in FIGS. 9 and 10, the fluid sterilizing apparatus 100b includes a first light source 10-1b, the chamber 30, a second light source 10-2b, and a control unit 50b. The first light source 10-1b includes the first light-transmitting member 40-1. The second light source 10-2b includes the second light-transmitting member 40-2. FIG. 10 illustrates a cross section of the fluid sterilizing apparatus 100b including the first light source 10-1b, the chamber 30, and the second light source 10-2b. In this regard, the same applies to FIGS. 11 to 14.

The first light source 10-1b includes the first optoelectronic element 11-1, the first optoelectronic element for emitted light 12-1, the third optoelectronic element 11-3, and a third optoelectronic element for emitted light 12-3. The second light source 10-2b includes a second optoelectronic element 11-2, the second optoelectronic element for emitted light 12-2, a fourth optoelectronic element 11-4, and a fourth optoelectronic element for emitted light 12-4. The first light source 10-1 b and the second light source 10-2 b are disposed to face each other.

In the fluid sterilizing apparatus 100b, the control unit 50b includes the processor 53 configured to sequentially switch between the first state, the second state, the third state, and the fourth state. The first state is a state in which the first optoelectronic element 11-1 emits ultraviolet light, and the optoelectronic elements other than the first optoelectronic element 11-1 do not emit ultraviolet light. The second state is a state in which the third optoelectronic element 11-3 emits ultraviolet light, and the optoelectronic elements other than the third optoelectronic element 11-3 do not emit ultraviolet light. The third state is a state in which the second optoelectronic element 11-2 emits ultraviolet light, and the optoelectronic elements other than the second optoelectronic element 11-2 do not emit ultraviolet light. The fourth state is a state in which the fourth optoelectronic element 11-4 emits ultraviolet light, and the optoelectronic elements other than the fourth optoelectronic element 11-4 do not emit ultraviolet light.

In the first state, the third optoelectronic element 11-3 outputs the third reflected light reception signal. The first optoelectronic element for emitted light 12-1 outputs the first emitted light reception signal. The second optoelectronic element 11-2 or the fourth optoelectronic element 11-4 outputs the first transmitted light reception signal. The control unit 50b controls the emission of the ultraviolet light from the first optoelectronic element 11-1 based on the third reflected light reception signal, the first emitted light reception signal, and the first transmitted light reception signal.

In the second state, the first optoelectronic element 11-1 outputs the first reflected light reception signal. The third optoelectronic element for emitted light 12-3 outputs the third emitted light reception signal. The second optoelectronic element 11-2 or the fourth optoelectronic element 11-4 outputs the third transmitted light reception signal. The control unit 50b controls the emission of the ultraviolet light from the third optoelectronic element 11-3 based on the first reflected light reception signal, the third emitted light reception signal, and the third transmitted light reception signal.

In the third state, the fourth optoelectronic element 11-4 outputs the fourth reflected light reception signal. The second optoelectronic element for emitted light 12-2 outputs the second emitted light reception signal. The first optoelectronic element 11-1 or the third optoelectronic element 11-3 outputs the second transmitted light reception signal. The control unit 50b controls the emission of the ultraviolet light from the second optoelectronic element 11-2 based on the fourth reflected light reception signal, the second emitted light reception signal, and the second transmitted light reception signal.

In the fourth state, the second optoelectronic element 11-2 outputs the second reflected light reception signal. The fourth optoelectronic element for emitted light 12-4 outputs the fourth emitted light reception signal. The first optoelectronic element 11-1 or the third optoelectronic element 11-3 outputs the fourth transmitted light reception signal. The control unit 50b controls the emission of the ultraviolet light from the fourth optoelectronic element 11-4 based on the second reflected light reception signal, the fourth emitted light reception signal, and the fourth transmitted light reception signal.

The fluid sterilizing apparatus 100b sequentially emits ultraviolet light from the first optoelectronic element 11-1 in the first state, the second optoelectronic element 11-2 in the third state, the third optoelectronic element 11-3 in the second state, and the fourth optoelectronic element 11-4 in the fourth state. In each of these states, no ultraviolet light is emitted from the optoelectronic elements other than the optoelectronic element emitting the ultraviolet light. Thus, the fluid sterilizing apparatus 100b can reduce an increase in the junction temperature of the first optoelectronic element 11-1, the second optoelectronic element 11-2, the third optoelectronic element 11-3, and the fourth optoelectronic element 11-4. This extends the lifetime of the first optoelectronic element 11-1, the second optoelectronic element 11-2, the third optoelectronic element 11-3, or the fourth optoelectronic element 11-4, and thus extends a cycle of replacement of the first optoelectronic element 11-1, the second optoelectronic element 11-2, the third optoelectronic element 11-3, or the fourth optoelectronic element 11-4. Because the cycle of replacement of the first optoelectronic element 11-1, the second optoelectronic element 11-2, the third optoelectronic element 11-3, or the fourth optoelectronic element 11-4 is extended, it is possible to improve maintainability of the fluid sterilizing apparatus 100b in the third embodiment of the present disclosure. Also, by causing the first optoelectronic element 11-1, the second optoelectronic element 11-2, the third optoelectronic element 11-3, and the fourth optoelectronic element 11-4 to function as an optoelectronic element for transmitted light, it is possible to reduce the number of parts of the fluid sterilizing apparatus 100b, thereby enabling achieving a simplified configuration and a reduced cost in the third embodiment of the present disclosure. Note that the transition sequence between the first state, the second state, the third state, and the fourth state may follow any order, as configured.

The above is a main difference of the fluid sterilizing apparatus 100b from the fluid sterilizing apparatus 100 according to the first embodiment of the present disclosure.

Hereinafter, the fluid sterilizing apparatus 100b will be described in more detail.

### (First Light Source 10-1b)

In the example illustrated in FIGS. 10 to 15, the first light source 10-1b includes the first optoelectronic element 11-1, the first optoelectronic element for emitted light 12-1, the third optoelectronic element 11-3, and the third optoelectronic element for emitted light 12-3. Also, the first light source 10-1b includes the first light-emitting unit 15-1 and the third light-emitting unit 15-3 that are disposed over the first substrate 14-1.

The third light-emitting unit 15-3 includes a third housing 151-3 and a third cover 152-3. The third optoelectronic element 11-3 and the third optoelectronic element for emitted light 12-3 are housed in the third housing 151-3. The third housing 151-3 supports the third cover 152-3. The third housing 151-3 and the third cover 152-3 hermetically seal the third optoelectronic element 11-3 and the third optoelectronic element for emitted light 12-3.

The third cover 152-3 is disposed to face the third optoelectronic element 11-3 and the third optoelectronic element for emitted light 12-3, and disposed between: the third optoelectronic element 11-3 and the third optoelectronic element for emitted light 12-3; and the first light-transmitting member 40-1. The third cover 152-3 transmits a part of the ultraviolet light emitted from the third optoelectronic element 11-3. The third cover 152-3 includes a third light quantity adjusting member 153-3. The third light quantity adjusting member 153-3 is disposed to face the third optoelectronic element for emitted light 12-3.

The third housing 151-3 can be configured in the same manner as in the first housing 151-1. The third cover 152-3 can be configured in the same manner as in the first cover 152-1.

The third optoelectronic element 11-3 can be configured in the same manner as in the first optoelectronic element 11-1. The third optoelectronic element for emitted light 12-3 can be configured in the same manner as in the first optoelectronic element for emitted light 12-1. The third light-emitting unit 15-3 can be configured in the same manner as in the first light-emitting unit 15-1.

### (First Light-Transmitting Member 40-1)

The first light-transmitting member 40-1 is disposed between: the first optoelectronic element 11-1, the third optoelectronic element 11-3, the first optoelectronic element for emitted light 12-1, and the third optoelectronic element for emitted light 12-3; and the inner space of the chamber 30.

### (Second Light Source 10-2b)

In the example illustrated in FIG. 10, the second light source 10-2b includes the second optoelectronic element 11-2, the second optoelectronic element for emitted light 12-2, the fourth optoelectronic element 11-4, and the fourth optoelectronic element for emitted light 12-4. Also, the second light source 10-2 b includes a second light-emitting unit 15-2 and a fourth light-emitting unit 15-4 that are disposed below the second substrate 14-2.

The second light-emitting unit 15-2 includes a second housing 151-2 and a second cover 152-2. The second optoelectronic element 11-2 and the second optoelectronic element for emitted light 12-2 are housed in the second housing 151-2. The second housing 151-2 supports the second cover 152-2. The second housing 151-2 and the second cover 152-2 hermetically seal the second optoelectronic element 11-2 and the second optoelectronic element for emitted light 12-2.

The fourth light-emitting unit 15-4 includes a fourth housing 151-4 and a fourth cover 152-4. The fourth optoelectronic element 11-4 and the fourth optoelectronic element for emitted light 12-4 are housed in the fourth housing 151-4. The fourth housing 151-4 supports the fourth cover 152-4. The fourth housing 151-4 and the fourth cover 152-4 hermetically seal the fourth optoelectronic element 11-4 and the fourth optoelectronic element for emitted light 12-4.

The second optoelectronic element 11-2, the second optoelectronic element for emitted light 12-2, the fourth optoelectronic element 11-4, and the fourth optoelectronic element for emitted light 12-4 are disposed to face the first optoelectronic element 11-1, the first optoelectronic element for emitted light 12-1, the third optoelectronic element 11-3, and the third optoelectronic element for emitted light 12-3.

Similar to the second light source 10-2, the second light source 10-2b can be removed from the fluid sterilizing apparatus 100b. The ability to remove the second light source 10-2b from the fluid sterilizing apparatus 100b facilitates removal of the contaminants adhering to the second light-transmitting member 40-2, replacement of the second substrate 14-2 over which the second light-emitting unit 15-2 and the fourth light-emitting unit 15-4 are placed, or the like.

The second cover 152-2 is disposed to face the second optoelectronic element 11-2 and the second optoelectronic element for emitted light 12-2, and disposed between: the second optoelectronic element 11-2 and the second optoelectronic element for emitted light 12-2; and the second light-transmitting member 40-2. The second cover 152-2 transmits a part of the ultraviolet light emitted from the second optoelectronic element 11-2. The second cover 152-2 includes a second light quantity adjusting member 153-2. The second light quantity adjusting member 153-2 is disposed to face the second optoelectronic element for emitted light 12-2.

The fourth cover 152-4 is disposed to face the fourth optoelectronic element 11-4 and the fourth optoelectronic element for emitted light 12-4, and disposed between: the fourth optoelectronic element 11-4 and the fourth optoelectronic element for emitted light 12-4; and the second light-transmitting member 40-2. The fourth cover 152-4 transmits a part of the ultraviolet light emitted from the fourth optoelectronic element 11-4. The fourth cover 152-4 includes a fourth light quantity adjusting member 153-4. The fourth light quantity adjusting member 153-4 is disposed to face the fourth optoelectronic element for emitted light 12-4.

The second housing 151-2 can be configured in the same manner as in the first housing 151-1. The second cover 152-2 can be configured in the same manner as in the first cover 152-1.

The second optoelectronic element 11-2 can be configured in the same manner as in the first optoelectronic element 11-1. The second optoelectronic element for emitted light 12-2 can be configured in the same manner as in the first optoelectronic element for emitted light 12-1. The second light-emitting unit 15-2 can be configured in the same manner as in the first light-emitting unit 15-1.

The fourth housing 151-4 can be configured in the same manner as in the first housing 151-1. The fourth cover 152-4 can be configured in the same manner as in the first cover 152-1.

The fourth optoelectronic element 11-4 can be configured in the same manner as in the first optoelectronic element 11-1. The fourth optoelectronic element for emitted light 12-4 can be configured in the same manner as in the first optoelectronic element for emitted light 12-1. The fourth light-emitting unit 15-4 can be configured in the same manner as in the first light-emitting unit 15-1.

### (Second Light-Transmitting Member 40-2)

The second light-transmitting member 40-2 is disposed between: the second optoelectronic element 11-2, the fourth optoelectronic element 11-4, the second optoelectronic element for emitted light 12-2, and the fourth optoelectronic element for emitted light 12-4; and the inner space of the chamber 30.

### (Chamber 30)

The fluid F located in the inner space of the chamber 30 is disposed between: the first optoelectronic element 11-1, the third optoelectronic element 11-3, the first optoelectronic element for emitted light 12-1, and the third optoelectronic element for emitted light 12-3; and the second optoelectronic element 11-2, the fourth optoelectronic element 11-4, the second optoelectronic element for emitted light 12-2, and the fourth optoelectronic element for emitted light 12-4. The fluid F located in the inner space of the chamber 30 is irradiated with the ultraviolet light emitted from the first optoelectronic element 11-1, the third optoelectronic element 11-3, the second optoelectronic element 11-2, and the fourth optoelectronic element 11-4.

### (Control Unit 50b)

The control unit 50b is used to sequentially switch between the first state, the second state, the third state, and the fourth state. In the example illustrated in FIG. 9, the control unit 50b includes a forward bias power supply 51A and a reverse bias power supply 51B. The example illustrated in FIG. 9 is the third state.

The control unit 50b switches application of the drive current, supplied from the forward bias power supply 51A, to any one of the first optoelectronic element 11-1, the second optoelectronic element 11-2, the third optoelectronic element 11-3, or the fourth optoelectronic element 11-4. Thus, the control unit 50b can sequentially switch between the first state, the second state, the third state, and the fourth state. Further, the control unit 50b can apply a reverse bias, from the reverse bias power supply 51B, to the first optoelectronic element 11-1, the second optoelectronic element 11-2, the third optoelectronic element 11-3, and the fourth optoelectronic element 11-4. Thus, the control unit 50b can cause the optoelectronic element, to which the reverse bias is applied, to function as a light-receiving element.

### (First State)

The first state is a state in which the first optoelectronic element 11-1 emits ultraviolet light, and the third optoelectronic element 11-3, the first optoelectronic element for emitted light 12-1, the third optoelectronic element for emitted light 12-3, the second optoelectronic element 11-2, the fourth optoelectronic element 11-4, the second optoelectronic element for emitted light 12-2, and the fourth optoelectronic element for emitted light 12-4 do not emit ultraviolet light.

In the example illustrated in FIG. 11, in the first state, the third optoelectronic element 11-3 outputs the third reflected light reception signal upon receiving the ultraviolet light emitted from the first optoelectronic element 11-1 and reflected by the first light-transmitting member 40-1. The first optoelectronic element for emitted light 12-1 outputs the first emitted light reception signal upon receiving a part of the ultraviolet light emitted from the first optoelectronic element 11-1. The second optoelectronic element 11-2 or the fourth optoelectronic element 11-4 outputs the first transmitted light reception signal upon receiving the ultraviolet light emitted from the first optoelectronic element 11-1 and subsequently transmitted through the first light-transmitting member 40-1, the fluid F, and the second light-transmitting member 40-2. At least one of the second optoelectronic element 11-2 or the fourth optoelectronic element 11-4 can output the first transmitted light reception signal upon receiving the ultraviolet light emitted from the first optoelectronic element 11-1 and subsequently transmitted through the first light-transmitting member 40-1, the fluid F, and the second light-transmitting member 40-2. The control unit 50b controls the emission of the ultraviolet light from the first optoelectronic element 11-1 based on the third reflected light reception signal, the first emitted light reception signal, and the first transmitted light reception signal.

### (Second State)

The second state is a state in which the third optoelectronic element 11-3 emits ultraviolet light, and the first optoelectronic element 11-1, the first optoelectronic element for emitted light 12-1, the third optoelectronic element for emitted light 12-3, the second optoelectronic element 11-2, the fourth optoelectronic element 11-4, the second optoelectronic element for emitted light 12-2, and the fourth optoelectronic element for emitted light 12-4 do not emit ultraviolet light.

In the example illustrated in FIG. 12, in the second state, the first optoelectronic element 11-1 outputs the first reflected light reception signal upon receiving the ultraviolet light emitted from the third optoelectronic element 11-3 and reflected by the first light-transmitting member 40-1. The third light-emitting optoelectronic element 12-3 outputs the third transmitted light reception signal upon receiving a part of the ultraviolet light emitted from the third optoelectronic element 11-3. The second optoelectronic element 11-2 or the fourth optoelectronic element 11-4 outputs the third transmitted light reception signal upon receiving the ultraviolet light emitted from the third optoelectronic element 11-3 and subsequently transmitted through the first light-transmitting member 40-1, the fluid F, and the second light-transmitting member 40-2. At least one of the second optoelectronic element 11-2 or the fourth optoelectronic element 11-4 can output the third transmitted light reception signal upon receiving the ultraviolet light emitted from the third optoelectronic element 11-3 and subsequently transmitted through the first light-transmitting member 40-1, the fluid F, and the second light-transmitting member 40-2. The control unit 50b controls the emission of the ultraviolet light from the third optoelectronic element 11-3 based on the first reflected light reception signal, the third emitted light reception signal, and the third transmitted light reception signal.

### (Third State)

The third state is a state in which the second optoelectronic element 11-2 emits ultraviolet light, and the first optoelectronic element 11-1, the third optoelectronic element 11-3, the first optoelectronic element for emitted light 12-1, the third optoelectronic element for emitted light 12-3, the fourth optoelectronic element 11-4, the second optoelectronic element for emitted light 12-2, and the fourth optoelectronic element for emitted light 12-4 do not emit ultraviolet light.

In the example illustrated in FIG. 13, in the third state, the fourth optoelectronic element 11-4 outputs the fourth reflected light reception signal upon receiving the ultraviolet light emitted from the second optoelectronic element 11-2 and reflected by the second light-transmitting member 40-2. The second optoelectronic element for emitted light 12-2 outputs the second emitted light reception signal upon receiving a part of the ultraviolet light emitted from the second optoelectronic element 11-2. The first optoelectronic element 11-1 or the third optoelectronic element 11-3 outputs the second transmitted light reception signal upon receiving the ultraviolet light emitted from the second optoelectronic element 11-2 and subsequently transmitted through the second light-transmitting member 40-2, the fluid F, and the first light-transmitting member 40-1. At least one of the first optoelectronic element 11-1 or the third optoelectronic element 11-3 can output the second transmitted light reception signal upon receiving the ultraviolet light emitted from the second optoelectronic element 11-2 and subsequently transmitted through the second light-transmitting member 40-2, the fluid F, and the first light-transmitting member 40-1. The control unit 50b controls the emission of the ultraviolet light from the second optoelectronic element 11-2 based on the fourth reflected light reception signal, the second emitted light reception signal, and the second transmitted light reception signal.

### (Fourth State)

The fourth state is a state in which the fourth optoelectronic element 11-4 emits ultraviolet light, and the first optoelectronic element 11-1, the third optoelectronic element 11-3, the first optoelectronic element for emitted light 12-1, the third optoelectronic element for emitted light 12-3, the second optoelectronic element 11-2, the second optoelectronic element for emitted light 12-2, and the fourth optoelectronic element for emitted light 12-4 do not emit ultraviolet light.

In the example illustrated in FIG. 14, in the fourth state, the second optoelectronic element 11-2 outputs the second reflected light reception signal upon receiving the ultraviolet light emitted from the fourth optoelectronic element 11-4 and reflected by the second light-transmitting member 40-2. The fourth optoelectronic element for emitted light 12-4 outputs the fourth emitted light reception signal upon receiving a part of the ultraviolet light emitted from the fourth optoelectronic element 11-4. The first optoelectronic element 11-1 or the third optoelectronic element 11-3 outputs the fourth transmitted light reception signal upon receiving the ultraviolet light emitted from the fourth optoelectronic element 11-4 and subsequently transmitted through the second light-transmitting member 40-2, the fluid F, and the first light-transmitting member 40-1. At least one of the first optoelectronic element 11-1 or the third optoelectronic element 11-3 can output the fourth transmitted light reception signal upon receiving the ultraviolet light emitted from the fourth optoelectronic element 11-4 and subsequently transmitted through the second light-transmitting member 40-2, the fluid F, and the first light-transmitting member 40-1. The control unit 50b controls the emission of the ultraviolet light from the fourth optoelectronic element 11-4 based on the second reflected light reception signal, the fourth emitted light reception signal, and the fourth transmitted light reception signal.

The first emitted light reception signal includes information of the output of light emitted from the first optoelectronic element 11-1. The second emitted light reception signal includes information of the output of light emitted from the second optoelectronic element 11-2. The third emitted light reception signal includes information of the output of light emitted from the third optoelectronic element 11-3. The fourth emitted light reception signal includes information of the output of light emitted from the fourth optoelectronic element 11-4. The first emitted light reception signal, the second emitted light reception signal, the third emitted light reception signal, and the fourth emitted light reception signal are not impacted by the first light-transmitting member 40-1, the contaminants adhering to the first light-transmitting member 40-1, the second light-transmitting member 40-2, the contaminants adhering to the second light-transmitting member 40-2, the fluid F, or the like.

The first reflected light reception signal and the third reflected light reception signal account for effects, such as light absorption, scattering, or the like, caused by at least one of the first cover 152-1, the third cover 152-3, the first light-transmitting member 40-1, or the contaminants adhering to the first light-transmitting member 40-1. The second reflected light reception signal and the fourth reflected light reception signal account for effects, such as light absorption, scattering, or the like, caused by at least one of the second cover 152-2, the fourth cover 152-4, the second light-transmitting member 40-2, or the contaminants adhering to the second light-transmitting member 40-2.

The first transmitted light reception signal accounts for effects, such as light absorption, scattering, or the like, caused by at least one of the first cover 152-1, the first light-transmitting member 40-1, the contaminants adhering to the first light-transmitting member 40-1, the fluid F, the contaminants adhering to the second light-transmitting member 40-2, the second light-transmitting member 40-2, the second cover 152-2, or the fourth cover 152-4. The second transmitted light reception signal accounts for effects, such as light absorption, scattering, or the like, caused by at least one of the second cover 152-2, the first light-transmitting member 40-1, the contaminants adhering to the first light-transmitting member 40-1, the fluid F, the contaminants adhering to the second light-transmitting member 40-2, the second light-transmitting member 40-2, the first cover 152-1, or the third cover 152-3. The third transmitted light reception signal accounts for effects, such as light absorption, scattering, or the like, caused by at least one of the third cover 152-3, the first light-transmitting member 40-1, the contaminants adhering to the first light-transmitting member 40-1, the fluid F, the contaminants adhering to the second light-transmitting member 40-2, the second light-transmitting member 40-2, the second cover 152-2, or the fourth cover 152-4. The fourth transmitted light reception signal accounts for effects, such as light absorption, scattering, or the like, caused by at least one of the fourth cover 152-4, the first light-transmitting member 40-1, the contaminants adhering to the first light-transmitting member 40-1, the fluid F, the contaminants adhering to the second light-transmitting member 40-2, the second light-transmitting member 40-2, the first cover 152-1, or the third cover 152-3.

In the third embodiment of the present disclosure, the memory 52 can store at least the first transmitted light reception signal initial value, the second transmitted light reception signal initial value, the third transmitted light reception signal initial value, and the fourth transmitted light reception signal initial value. The fluid sterilizing apparatus 100b supplies, to the first optoelectronic element 11-1, a drive current enough to apply a predetermined dose of ultraviolet light to the fluid F, from the forward bias power supply 51A of the control unit 50, in a state in which no contaminant adheres to the first light-transmitting member 40-1 and the second light-transmitting member 40-2. The fluid sterilizing apparatus 100b stores, in the memory 52, the magnitude of the first transmitted light reception signal obtained at this time as the first transmitted light reception signal initial value. In this state, the fluid sterilizing apparatus 100b supplies, to the second optoelectronic element 11-2, a drive current enough to apply a predetermined dose of ultraviolet light to the fluid F, from the forward bias power supply 51A, and stores, in the memory 52, the magnitude of the second transmitted light reception signal obtained at this time as the second transmitted light reception signal initial value. Further, in this state, the fluid sterilizing apparatus 100b supplies, to the third optoelectronic element 11-3, a drive current enough to apply a predetermined dose of ultraviolet light to the fluid F, from the forward bias power supply 51A, and stores, in the memory 52, the magnitude of the third transmitted light reception signal obtained at this time as the third transmitted light reception signal initial value. Further, in this state, the fluid sterilizing apparatus 100b supplies, to the fourth optoelectronic element 11-4, a drive current enough to apply a predetermined dose of ultraviolet light to the fluid F, from the forward bias power supply 51A, and stores, in the memory 52, the magnitude of the fourth transmitted light reception signal obtained at this time as the fourth transmitted light reception signal initial value.

In the first state, the operation of the fluid sterilizing apparatus 100b when the control unit 50b outputs the first error signal and the second error signal is the same as the operation of the fluid sterilizing apparatus 100 in the first embodiment except that the first reflected light reception signal is replaced with the third reflected light reception signal. In the second state, the operation of the fluid sterilizing apparatus 100b when the control unit 50b outputs the first error signal and the second error signal is the same as the operation of the fluid sterilizing apparatus 100 in the first embodiment except that the first emitted light reception signal is replaced with the third emitted light reception signal, and the first transmitted light reception signal is replaced with the third transmitted light reception signal. In the third state, the operation of the fluid sterilizing apparatus 100b when the control unit 50b outputs the first error signal and the second error signal is the same as the operation of the fluid sterilizing apparatus 100 in the first embodiment except that the first emitted light reception signal is replaced with the second emitted light reception signal, the first reflected light reception signal is replaced with the fourth reflected light reception signal, and the first transmitted light reception signal is replaced with the second transmitted light reception signal. In the fourth state, the operation of the fluid sterilizing apparatus 100b when the control unit 50b outputs the first error signal and the second error signal is the same as the operation of the fluid sterilizing apparatus 100 in the first embodiment except that the first emitted light reception signal is replaced with the fourth emitted light reception signal, the first reflected light reception signal is replaced with the second reflected light reception signal, and the first transmitted light reception signal is replaced with the fourth transmitted light reception signal.

<Effects of Fluid Sterilizing Apparatus 100b>

Effects of the fluid sterilizing apparatus 100b other than those described above are the same as the effects of the fluid sterilizing apparatus 100 described in the first embodiment of the present disclosure.

### Fourth Embodiment

Next, a fluid sterilizing apparatus according to a fourth embodiment of the present disclosure will be described.

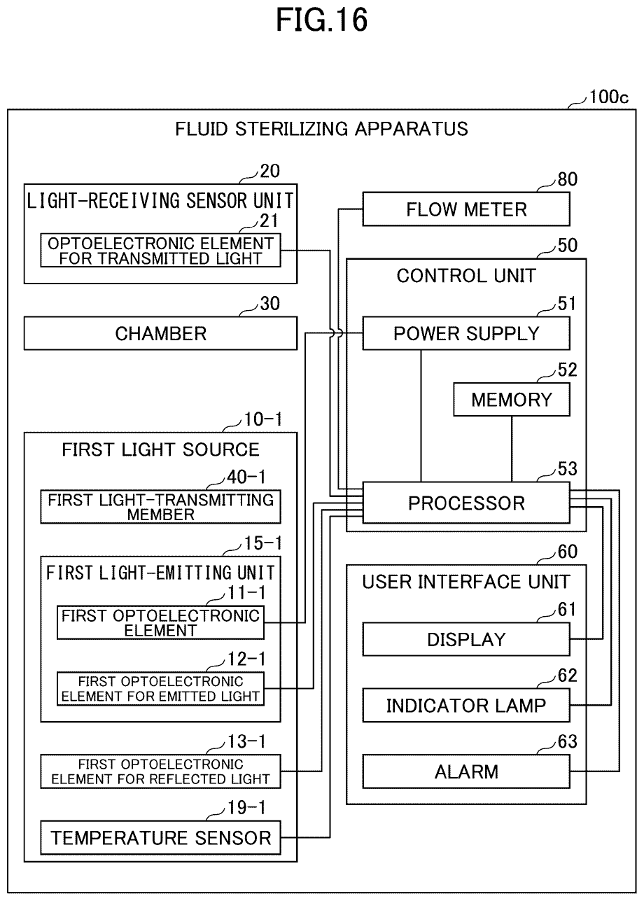

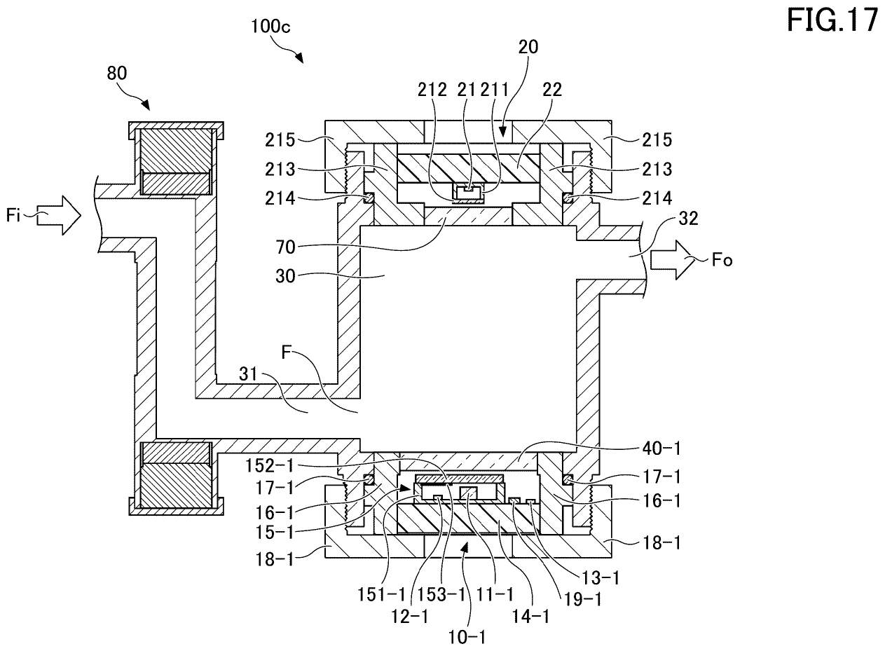

FIG. 16 is a block diagram illustrating an overall configuration of a fluid sterilizing apparatus 100c according to the fourth embodiment of the present disclosure. FIG. 17 is a schematic cross-sectional view illustrating a configuration of the fluid sterilizing apparatus 100c in the vicinity of the chamber 30 included in the fluid sterilizing apparatus 100c.

The fourth embodiment is mainly different from the first embodiment in that the fluid sterilizing apparatus 100c further includes a flow meter 80, and the processor 53 controls emission of ultraviolet light from the first optoelectronic element 11-1 upon further receiving a flow signal from the flow meter 80.

When a flow rate of the fluid fluctuates, as illustrated in FIGS. 16 and 17, the fluid sterilizing apparatus 100c can further include the flow meter 80 configured to obtain a predetermined dose of ultraviolet light applied to the fluid to be sterilized. Also, in the fluid sterilizing apparatus 100c, the processor 53 included in the control unit 50 can control the illuminance of the ultraviolet light emitted from the first light source 10-1 upon further receiving the flow signal from the flow meter 80.

In the example illustrated in FIG. 17, the flow meter 80 is disposed at the inlet 31 of the chamber 30. The flow meter 80 is configured to measure a flow rate of the fluid flowing through the chamber 30, and output a flow signal to the control unit 50. The processor 53 controls emission of the ultraviolet light from the first optoelectronic element 11-1 upon receiving the first reflected light reception signal, the first emitted light reception signal, the first transmitted light reception signal, and the flow signal from the flow meter 80. For example, the processor 53 can control the emission of the ultraviolet light from the first optoelectronic element 11-1 such that the higher flow rate the flow signal indicates, the higher the illuminance of the ultraviolet light emitted from the first light source 10-1 becomes.

An ultrasonic flow meter or the like can be used as the flow meter 80. However, another type of a flow meter, such as a volumetric flow meter, a differential pressure flow meter, or the like may be used as the flow meter 80. The flow meter 80 may be disposed at any position different from the inlet 31, and may be disposed at the outlet 32. Alternatively, the flow meter 80 may be disposed at both the inlet 31 and the outlet 32. The number of the flow meter 80 is not limited to one, and may be two or more.

As illustrated in FIG. 17, the first substrate 14-1 may further include a temperature sensor 19-1 in the vicinity of a region to which the first light-emitting unit 15-1 is to be connected. In this case, when the control unit 50 controls irradiation of the ultraviolet light from the first optoelectronic element 11-1, the processor 53 included in the control unit 50 receives a temperature signal output from the temperature sensor, i.e., the processor 53 included in the control unit 50 can use the temperature signal for control of the emission of the ultraviolet light from the first optoelectronic element 11-1.

FIGS. 16 and 17 illustrate an example in which the fourth embodiment is combined with the first embodiment. However, the fourth embodiment can be combined with the second embodiment or the third embodiment.

Although the embodiments of the present disclosure have been described above in detail, the above-described embodiments are by no means limitations. Various modifications and substitutions can be made to the above-described embodiments without departing from the scope of the claims recited.

All numerical values, such as ordinals, numbers, quantities, and the like, used in the description of the embodiments are merely examples for specifically describing the technique of the present disclosure, and the present disclosure is not limited to the exemplified numerical values. Further, the connection relationship between the components is merely an example for specifically describing the technique of the present disclosure, and a connection relationship achieving the functions of the present disclosure is not limited to this.

The fluid sterilizing apparatus of the present disclosure can irradiate a fluid with a required dose of ultraviolet light, and therefore is suitably used as an apparatus intended to sterilize water, air, or the like. The light source and light source apparatus of the present disclosure can irradiate a fluid with a required dose of ultraviolet light, and therefore are suitably used as a light-exposing apparatus, a printing apparatus, such as an inkjet printer or the like, a sterilizing apparatus, a bonding apparatus, or the like, each using ultraviolet light. Further, the light source and light source apparatus of the present disclosure can irradiate a fluid with a required dose of ultraviolet light while mitigating the impact of the contaminants on a light-transmitting member, such as a glass plate or the like. Thus, the light source and light source apparatus of the present disclosure are particularly preferably used for applications as a fluid sterilizing apparatus.

The embodiments of the present disclosure may include, for example, the following.

### <Clause 1>

A fluid sterilizing apparatus, including:
a first optoelectronic element, a first optoelectronic element for emitted light, and a first optoelectronic element for reflected light;
an optoelectronic element for transmitted light disposed to face the first optoelectronic element, the first optoelectronic element for emitted light, and the first optoelectronic element for reflected light;
a chamber including an inner space that allows passage of a fluid to be irradiated with ultraviolet light emitted from the first optoelectronic element, wherein
   the chamber is disposed between the first optoelectronic element and the optoelectronic element for transmitted light,
   the chamber is disposed between the first optoelectronic element for emitted light and the optoelectronic element for transmitted light, and
   the chamber is disposed between the first optoelectronic element for reflected light and the optoelectronic element for transmitted light;
a first light-transmitting member, wherein
   the first light-transmitting member is disposed between the first optoelectronic element and the inner space of the chamber,
   the first light-transmitting member is disposed between the first optoelectronic element for emitted light and the inner space of the chamber, and
   the first light-transmitting member is disposed between the first optoelectronic element for reflected light and the inner space of the chamber; and
a control unit including a processor configured to control emission of the ultraviolet light from the first optoelectronic element upon receiving
   a first reflected light reception signal generated from the first optoelectronic element for reflected light upon receiving the ultraviolet light emitted from the first optoelectronic element and reflected by the first light-transmitting member,
   a first emitted light reception signal generated from the first optoelectronic element for emitted light upon receiving a part of the ultraviolet light emitted from the first optoelectronic element, and
   a first transmitted light reception signal generated from the optoelectronic element for transmitted light upon receiving the ultraviolet light emitted from the first optoelectronic element and subsequently transmitted through the first light-transmitting member and the fluid.

### <Clause 2>

The fluid sterilizing apparatus according to clause 1, wherein
the first optoelectronic element is configured to emit the ultraviolet light in accordance with a drive current supplied to the first optoelectronic element, and
the processor is configured to
   control a dose of ultraviolet light applied to the fluid from the first optoelectronic element to be within a predetermined range by adjusting the drive current supplied to the first optoelectronic element.

### <Clause 3>

The fluid sterilizing apparatus according to clause 2, wherein
the control unit includes a memory storing at least a first transmitted light reception signal initial value and a predetermined transmitted light threshold, and
the processor is configured to
   output a first error signal in a case that an absolute value of a difference between a magnitude of the first transmitted light reception signal and a magnitude of the first transmitted light reception signal initial value exceeds the predetermined transmitted light threshold.

### <Clause 4>

The fluid sterilizing apparatus according to clause 3, wherein
the memory further stores a predetermined current threshold for the drive current, and
the processor is configured to
   output a second error signal indicating degradation in the first optoelectronic element in a case that the absolute value of the difference between the magnitude of the first transmitted light reception signal and the magnitude of the first transmitted light reception signal initial value does not exceed the predetermined transmitted light threshold, and a magnitude of the drive current supplied to the first optoelectronic element exceeds the predetermined current threshold.

### <Clause 5>

The fluid sterilizing apparatus according to any one of clauses 1 to 4, further including:
a first cover that is disposed to face the first optoelectronic element, disposed between the first optoelectronic element and the first light-transmitting member, and transmits a part of the ultraviolet light emitted from the first optoelectronic element, wherein
   the first optoelectronic element for emitted light receives the ultraviolet light emitted from the first optoelectronic element and reflected by the first cover.

### <Clause 6>

The fluid sterilizing apparatus according to any one of clauses 1 to 4, further including:
a first cover that is disposed to face the first optoelectronic element, disposed between the first optoelectronic element and the first light-transmitting member, and transmits a part of the ultraviolet light emitted from the first optoelectronic element, wherein
the first cover includes a first light quantity adjusting member, and the first light quantity adjusting member is disposed to face the first optoelectronic element for emitted light.

### <Clause 7>

The fluid sterilizing apparatus according to clause 5 or 6, further including:
a first housing that houses the first optoelectronic element and the first optoelectronic element for emitted light, and supports the first cover.

### <Clause 8>

The fluid sterilizing apparatus according to any one of clauses 1 to 7, wherein
the first optoelectronic element and the first optoelectronic element for reflected light are disposed to directly face the first light-transmitting member.

### <Clause 9>

A fluid sterilizing apparatus, including:
one or more first optoelectronic elements, a first optoelectronic element for emitted light, and a first optoelectronic element for reflected light;
one or more second optoelectronic elements, a second optoelectronic element for emitted light, and a second optoelectronic element for reflected light that are disposed to face the one or more first optoelectronic elements, the first optoelectronic element for emitted light, and the first optoelectronic element for reflected light;
a chamber including an inner space that allows passage of a fluid to be irradiated with ultraviolet light emitted from the one or more first optoelectronic elements, wherein
   the chamber is disposed between (A) and (B), wherein
      (A) is a combination of the one or more first optoelectronic elements, the first optoelectronic element for emitted light, and the first optoelectronic element for reflected light, and
      (B) is a combination of the one or more second optoelectronic elements, the second optoelectronic element for emitted light, and the second optoelectronic element for reflected light;
a first light-transmitting member, wherein
   the first light-transmitting member is disposed between the one or more first optoelectronic elements and the inner space of the chamber,
   the first light-transmitting member is disposed between the first optoelectronic element for emitted light and the inner space of the chamber, and
   the first light-transmitting member is disposed between the first optoelectronic element for reflected light and the inner space of the chamber;
a second light-transmitting member, wherein
   the second light-transmitting member is disposed between the one or more second optoelectronic elements and the inner space of the chamber,
   the second light-transmitting member is disposed between the second optoelectronic element for emitted light and the inner space of the chamber, and
   the second light-transmitting member is disposed between the second optoelectronic element for reflected light and the inner space of the chamber; and
a control unit including a processor configured to alternately switch between
   a state I in which the one or more first optoelectronic elements emit the ultraviolet light and the first optoelectronic element for emitted light, the first optoelectronic element for reflected light, the one or more second optoelectronic elements, the second optoelectronic element for emitted light, and the second optoelectronic element for reflected light do not emit the ultraviolet light, and
   a state II in which the one or more second optoelectronic elements emit the ultraviolet light and the one or more first optoelectronic elements, the first optoelectronic element for emitted light, the first optoelectronic element for reflected light, the second optoelectronic element for emitted light, and the second optoelectronic element for reflected light do not emit the ultraviolet light, wherein
   in the state I,
      the first optoelectronic element for reflected light outputs a first reflected light reception signal upon receiving the ultraviolet light emitted from the one or more first optoelectronic elements and reflected by the first light-transmitting member,
      the first optoelectronic element for emitted light outputs a first emitted light reception signal upon receiving a part of the ultraviolet light emitted from the one or more first optoelectronic elements,
      the second optoelectronic element for reflected light outputs a first transmitted light reception signal upon receiving the ultraviolet light emitted from the one or more first optoelectronic elements and subsequently transmitted through the first light-transmitting member, the fluid, and the second light-transmitting member, and
      the processor controls emission of the ultraviolet light from the one or more first optoelectronic elements based on the first reflected light reception signal, the first emitted light reception signal, and the first transmitted light reception signal, and
   in the state II,
      the second optoelectronic element for reflected light outputs a second reflected light reception signal upon receiving the ultraviolet light emitted from the one or more second optoelectronic elements and reflected by the second light-transmitting member,
      the second optoelectronic element for emitted light outputs a second emitted light reception signal upon receiving a part of the ultraviolet light emitted from the one or more second optoelectronic elements,
      the first optoelectronic element for reflected light outputs a second transmitted light reception signal upon receiving the ultraviolet light emitted from the one or more second optoelectronic elements and subsequently transmitted through the second light-transmitting member, the fluid, and the first light-transmitting member, and
      the processor controls emission of the ultraviolet light from the one or more second optoelectronic elements based on the second reflected light reception signal, the second emitted light reception signal, and the second transmitted light reception signal.

### <Clause 10>

The fluid sterilizing apparatus according to clause 9, wherein
two or more of the first optoelectronic elements are connected in parallel, and
two or more of the second optoelectronic elements are connected in parallel.

### <Clause 11>

The fluid sterilizing apparatus according to clause 9 or 10, wherein
the one or more first optoelectronic elements and the one or more second optoelectronic elements are configured to emit the ultraviolet light in accordance with a drive current supplied to the one or more first optoelectronic elements and the one or more second optoelectronic elements, and
the processor is configured to
   control a dose of ultraviolet light applied to the fluid from the one or more first optoelectronic elements or the one or more second optoelectronic elements to be within a predetermined range by adjusting the drive current supplied to the one or more first optoelectronic elements or the one or more second optoelectronic elements.

### <Clause 12>

The fluid sterilizing apparatus according to clause 11, wherein
the control unit includes a memory storing at least a first transmitted light reception signal initial value, a second transmitted light reception signal initial value, and a predetermined transmitted light threshold, and
the processor is configured to
   output a first error signal in a case that an absolute value of a difference between a magnitude of the first transmitted light reception signal and a magnitude of the first transmitted light reception signal initial value or an absolute value of a difference between a magnitude of the second transmitted light reception signal and a magnitude of the second transmitted light reception signal initial value exceeds the predetermined transmitted light threshold.

### <Clause 13>

The fluid sterilizing apparatus according to clause 12, wherein
the memory further stores a predetermined current threshold for the drive current, and
the processor is configured to
   output a second error signal indicating degradation in the one or more first optoelectronic elements or the one or more second optoelectronic elements
   in a case that the absolute value of the difference between the magnitude of the first transmitted light reception signal and the magnitude of the first transmitted light reception signal initial value does not exceed the predetermined transmitted light threshold, and a magnitude of the drive current supplied to the one or more first optoelectronic elements exceeds the predetermined current threshold; or
   in a case that the absolute value of the difference between the magnitude of the second transmitted light reception signal and the magnitude of the second transmitted light reception signal initial value does not exceed the predetermined transmitted light threshold, and a magnitude of the drive current supplied to the one or more second optoelectronic elements exceeds the predetermined current threshold.

### <Clause 14>

The fluid sterilizing apparatus according to any one of clauses 9 to 13, further including:
a first cover that is disposed to face the one or more first optoelectronic elements and the first optoelectronic element for emitted light, and transmits a part of the ultraviolet light emitted from the one or more first optoelectronic elements, wherein
   the first cover is disposed between the one or more first optoelectronic elements and the first light-transmitting member, and
   the first cover is disposed between the first optoelectronic element for emitted light and the first light-transmitting member; and
a second cover that is disposed to face the one or more second optoelectronic elements and the second optoelectronic element for emitted light, and transmits a part of the ultraviolet light emitted from the one or more second optoelectronic elements, wherein
   the second cover is disposed between the one or more second optoelectronic elements and the second light-transmitting member, and
   the second cover is disposed between the second optoelectronic element for emitted light and the second light-transmitting member, wherein
the first cover includes a first light quantity adjusting member, and the first light quantity adjusting member is disposed to face the first optoelectronic element for emitted light, and
the second cover includes a second light quantity adjusting member, and the second light quantity adjusting member is disposed to face the second optoelectronic element for emitted light.

### <Clause 15>

The fluid sterilizing apparatus according to clause 14, further including:
a first housing that houses the one or more first optoelectronic elements and the first optoelectronic element for emitted light, and supports the first cover, and
a second housing that houses the one or more second optoelectronic elements and the second optoelectronic element for emitted light, and supports the second cover.
<Clause 16>

A fluid sterilizing apparatus, including:
a first optoelectronic element, a third optoelectronic element, a first optoelectronic element for emitted light, and a third optoelectronic element for emitted light;
a second optoelectronic element, a fourth optoelectronic element, a second optoelectronic element for emitted light, and a fourth optoelectronic element for emitted light that are disposed to face the first optoelectronic element, the third optoelectronic element, the first optoelectronic element for emitted light, and the third optoelectronic element for emitted light;
a chamber including an inner space that allows passage of a fluid to be irradiated with ultraviolet light emitted from the first optoelectronic element, wherein
   the chamber is disposed between (A) and (B), wherein
      (A) is a combination of the first optoelectronic element, the third optoelectronic element, the first optoelectronic element for emitted light, and the third optoelectronic element for emitted light, and
      (B) is a combination of the second optoelectronic element, the fourth optoelectronic element, the second optoelectronic element for emitted light, and the fourth optoelectronic element for emitted light;
a first light-transmitting member, wherein
   the first light-transmitting member is disposed between the first optoelectronic element and the inner space of the chamber,
   the first light-transmitting member is disposed between the third optoelectronic element and the inner space of the chamber,
   the first light-transmitting member is disposed between the first optoelectronic element for emitted light and the inner space of the chamber, and
   the first light-transmitting member is disposed between the third optoelectronic element for emitted light and the inner space of the chamber;
a second light-transmitting member, wherein
   the second light-transmitting member is disposed between the second optoelectronic element and the inner space of the chamber,
   the second light-transmitting member is disposed between the fourth optoelectronic element and the inner space of the chamber,
   the second light-transmitting member is disposed between the second optoelectronic element for emitted light and the inner space of the chamber, and
   the second light-transmitting member is disposed between the fourth optoelectronic element for emitted light and the inner space of the chamber; and
a control unit including a processor configured to sequentially switch between
   a first state in which the first optoelectronic element emits the ultraviolet light, and the third optoelectronic element, the first optoelectronic element for emitted light, the third optoelectronic element for emitted light, the second optoelectronic element, the fourth optoelectronic element, the second optoelectronic element for emitted light, and the fourth optoelectronic element for emitted light do not emit the ultraviolet light,
   a second state in which the third optoelectronic element emits the ultraviolet light, and the first optoelectronic element, the first optoelectronic element for emitted light, the third optoelectronic element for emitted light, the second optoelectronic element, the fourth optoelectronic element, the second optoelectronic element for emitted light, and the fourth optoelectronic element for emitted light do not emit the ultraviolet light,
   a third state in which the second optoelectronic element emits the ultraviolet light, and the first optoelectronic element, the third optoelectronic element, the first optoelectronic element for emitted light, the third optoelectronic element for emitted light, the fourth optoelectronic element, the second optoelectronic element for emitted light, and the fourth optoelectronic element for emitted light do not emit the ultraviolet light, and
   a fourth state in which the fourth optoelectronic element emits the ultraviolet light, and the first optoelectronic element, the third optoelectronic element, the first optoelectronic element for emitted light, the third optoelectronic element for emitted light, the second optoelectronic element, the second optoelectronic element for emitted light, and the fourth optoelectronic element for emitted light do not emit the ultraviolet light, wherein
in the first state,
   the third optoelectronic element outputs a third reflected light reception signal upon receiving the ultraviolet light emitted from the first optoelectronic element and reflected by the first light-transmitting member,
   the first optoelectronic element for emitted light outputs a first emitted light reception signal upon receiving a part of the ultraviolet light emitted from the first optoelectronic element,
   the second optoelectronic element or the fourth optoelectronic element outputs a first transmitted light reception signal upon receiving the ultraviolet light emitted from the first optoelectronic element and subsequently transmitted through the first light-transmitting member, the fluid, and the second light-transmitting member, and
   the processor controls emission of the ultraviolet light from the first optoelectronic element based on the third reflected light reception signal, the first emitted light reception signal, and the first transmitted light reception signal, and
in the second state,
   the first optoelectronic element outputs a first reflected light reception signal upon receiving the ultraviolet light emitted from the third optoelectronic element and reflected by the first light-transmitting member,
   the third optoelectronic element for emitted light outputs a third emitted light reception signal upon receiving a part of the ultraviolet light emitted from the third optoelectronic element,
   the second optoelectronic element or the fourth optoelectronic element outputs a third transmitted light reception signal upon receiving the ultraviolet light emitted from the third optoelectronic element and subsequently transmitted through the first light-transmitting member, the fluid, and the second light-transmitting member, and
   the processor controls emission of the ultraviolet light from the third optoelectronic element based on the first reflected light reception signal, the third emitted light reception signal, and the third transmitted light reception signal,
in the third state,
   the fourth optoelectronic element outputs a fourth reflected light reception signal upon receiving the ultraviolet light emitted from the second optoelectronic element and reflected by the second light-transmitting member,
   the second optoelectronic element for emitted light outputs a second emitted light reception signal upon receiving a part of the ultraviolet light emitted from the second optoelectronic element,
   the first optoelectronic element or the third optoelectronic element outputs a second transmitted light reception signal upon receiving the ultraviolet light emitted from the second optoelectronic element and subsequently transmitted through the second light-transmitting member, the fluid, and the first light-transmitting member, and
   the processor controls emission of the ultraviolet light from the second optoelectronic element based on the fourth reflected light reception signal, the second emitted light reception signal, and the second transmitted light reception signal, and
in the fourth state,
   the second optoelectronic element outputs a second reflected light reception signal upon receiving the ultraviolet light emitted from the fourth optoelectronic element and reflected by the second light-transmitting member,
   the fourth optoelectronic element for emitted light outputs a fourth emitted light reception signal upon receiving a part of the ultraviolet light emitted from the fourth optoelectronic element,
   the first optoelectronic element or the third optoelectronic element outputs a fourth transmitted light reception signal upon receiving the ultraviolet light emitted from the fourth optoelectronic element and subsequently transmitted through the second light-transmitting member, the fluid, and the first light-transmitting member, and
   the processor controls emission of the ultraviolet light from the fourth optoelectronic element based on the second reflected light reception signal, the fourth emitted light reception signal, and the fourth transmitted light reception signal.

### <Clause 17>

The fluid sterilizing apparatus according to clause 16, wherein
the first optoelectronic element, the second optoelectronic element, the third optoelectronic element, and the fourth optoelectronic element are configured to emit the ultraviolet light in accordance with a drive current supplied to the first optoelectronic element, the second optoelectronic element, the third optoelectronic element, and the fourth optoelectronic element, and
the processor is configured to
   control a dose of ultraviolet light applied to the fluid from the first optoelectronic element, the second optoelectronic element, the third optoelectronic element, or the fourth optoelectronic element to be within a predetermined range by adjusting the drive current supplied to the first optoelectronic element, the second optoelectronic element, the third optoelectronic element, or the fourth optoelectronic element.

### <Clause 18>

The fluid sterilizing apparatus according to clause 17, wherein
the control unit includes a memory storing at least a first transmitted light reception signal initial value, a third transmitted light reception signal initial value, a second transmitted light reception signal initial value, a fourth transmitted light reception signal initial value, and a predetermined transmitted light threshold, and
the processor is configured to
   output a first error signal in a case that an absolute value of a difference between a magnitude of the first transmitted light reception signal and a magnitude of the first transmitted light reception signal initial value, an absolute value of a difference between a magnitude of the third transmitted light reception signal and a magnitude of the third transmitted light reception signal initial value, an absolute value of a difference between a magnitude of the second transmitted light reception signal and a magnitude of the second transmitted light reception signal initial value, or an absolute value of a difference between a magnitude of the fourth transmitted light reception signal and a magnitude of the fourth transmitted light reception signal initial value exceeds the predetermined transmitted light threshold.

### <Clause 19>

The fluid sterilizing apparatus according to clause 18, wherein
the memory further stores a predetermined current threshold for the drive current, and
the processor is configured to
   output a second error signal indicating degradation in the first optoelectronic element, the second optoelectronic element, the third optoelectronic element, or the fourth optoelectronic element
   in a case that the absolute value of the difference between the magnitude of the first transmitted light reception signal and the magnitude of the first transmitted light reception signal initial value does not exceed the predetermined transmitted light threshold, and a magnitude of the drive current supplied to the first optoelectronic element exceeds the predetermined current threshold;
   in a case that the absolute value of the difference between the magnitude of the third transmitted light reception signal and the magnitude of the third transmitted light reception signal initial value does not exceed the predetermined transmitted light threshold, and a magnitude of the drive current supplied to the third optoelectronic element exceeds the predetermined current threshold;
   in a case that the absolute value of the difference between the magnitude of the second transmitted light reception signal and the magnitude of the second transmitted light reception signal initial value does not exceed the predetermined transmitted light threshold, and a magnitude of the drive current supplied to the second optoelectronic element exceeds the predetermined current threshold; or
   in a case that the absolute value of the difference between the magnitude of the fourth transmitted light reception signal and the magnitude of the fourth transmitted light reception signal initial value does not exceed the predetermined transmitted light threshold, and a magnitude of the drive current supplied to the fourth optoelectronic element exceeds the predetermined current threshold.

### <Clause 20>

The fluid sterilizing apparatus according to clause 16, further comprising:
a first cover that is disposed to face the first optoelectronic element, disposed between the first optoelectronic element and the first light-transmitting member, and transmits a part of the ultraviolet light emitted from the first optoelectronic element;
a second cover that is disposed to face the second optoelectronic element, disposed between the second optoelectronic element and the second light-transmitting member, and transmits a part of the ultraviolet light emitted from the second optoelectronic element;
a third cover that is disposed to face the third optoelectronic element, disposed between the third optoelectronic element and the first light-transmitting member, and transmits a part of the ultraviolet light emitted from the third optoelectronic element; and
a fourth cover that is disposed to face the fourth optoelectronic element, disposed between the fourth optoelectronic element and the second light-transmitting member, and transmits a part of the ultraviolet light emitted from the fourth optoelectronic element, wherein
the first cover includes a first light quantity adjusting member, and the first light quantity adjusting member is disposed to face the first optoelectronic element for emitted light,
the second cover includes a second light quantity adjusting member, and the second light quantity adjusting member is disposed to face the second optoelectronic element for emitted light,
the third cover includes a third light quantity adjusting member, and the third light quantity adjusting member is disposed to face the third optoelectronic element for emitted light, and
the fourth cover includes a fourth light quantity adjusting member, and the fourth light quantity adjusting member is disposed to face the fourth optoelectronic element for emitted light.

### <Clause 21>

The fluid sterilizing apparatus according to any one of clauses 1 to 20, further including:
a flow meter, wherein
the processor controls the emission of the ultraviolet light from the first optoelectronic element upon further receiving a flow signal from the flow meter.

### <Clause 22>

A fluid sterilizing method performed by a fluid sterilizing apparatus, the fluid sterilizing method including:
irradiating a fluid located in an inner space of a chamber, disposed between a first optoelectronic element and an optoelectronic element for transmitted light disposed to face the first optoelectronic element, with ultraviolet light emitted from the first optoelectronic element;
outputting a first reflected light reception signal generated from a first optoelectronic element for reflected light upon receiving the ultraviolet light reflected by a first light-transmitting member disposed between the inner space of the chamber and the first optoelectronic element;
outputting a first emitted light reception signal generated from a first optoelectronic element for emitted light upon receiving a part of the ultraviolet light emitted from the first optoelectronic element;
outputting a first transmitted light reception signal generated from the optoelectronic element for transmitted light upon receiving the ultraviolet light emitted from the first optoelectronic element and subsequently transmitted through the first light-transmitting member and the fluid; and

controlling emission of the ultraviolet light from the first optoelectronic element by a control unit including a processor configured to receive the first reflected light reception signal, the first emitted light reception signal, and the first transmitted light reception signal.

### <Clause 23>

A light source, including:
a first light-emitting element;
a first light-receiving element for emitted light configured to receive a part of light from the first light-emitting element;
a first light-transmitting member configured to reflect a part of the light from the first light-emitting element; and
a first light-receiving element for reflected light configured to receive a part of the light reflected by the first light-transmitting member.

### <Clause 24>

The light source according to clause 23, further including:
a housing that houses the first light-receiving element and the first light-receiving element for emitted light; and
a cover joined with the housing, wherein
the cover is configured to reflect a part of the light from the first light-emitting element and transmit another part of the light from the first light-emitting element, and
the cover includes a light quantity adjusting member covering the first light-receiving element for emitted light.

### <Clause 25>

A light source apparatus, including:
a first optoelectronic element, a first optoelectronic element for emitted light, and a first optoelectronic element for reflected light;
a first light-transmitting member disposed to face the first optoelectronic element, the first optoelectronic element for emitted light, and the first optoelectronic element for reflected light; and
a control unit including a processor configured to
   control emission of light from the first optoelectronic element upon receiving
      a first reflected light reception signal generated from the first optoelectronic element for reflected light upon receiving light emitted from the first optoelectronic element and reflected by the first light-transmitting member, and
      a first emitted light reception signal generated from the first optoelectronic element for emitted light upon receiving a part of the light emitted from the first optoelectronic element.

### <Clause 26>

The light source apparatus according to clause 25, further including:
an optoelectronic element for transmitted light that is configured to output a first transmitted light reception signal upon receiving the light emitted from the first optoelectronic element and subsequently transmitted through the first light-transmitting member, wherein
the processor is configured to
   control the emission of the light from the first optoelectronic element based on the first reflected light reception signal, the first emitted light reception signal, and the first transmitted light reception signal.

### <Clause 27>

A light irradiating method performed by a light source apparatus that includes a first optoelectronic element, a first optoelectronic element for emitted light, and a first optoelectronic element for reflected light; a first light-transmitting member disposed to face the first optoelectronic element, the first optoelectronic element for emitted light, and the first optoelectronic element for reflected light; an optoelectronic element for transmitted light that is configured to output a first transmitted light reception signal upon receiving light emitted from the first optoelectronic element and subsequently transmitted through the first light-transmitting member; and a control unit including a processor, the light irradiating method including:
causing the processor to control emission of the light from the first optoelectronic element upon receiving
   a first reflected light reception signal generated from the first optoelectronic element for reflected light upon receiving the light emitted from the first optoelectronic element and reflected by the first light-transmitting member,
   a first emitted light reception signal generated from the first optoelectronic element for emitted light upon receiving a part of the light emitted from the first optoelectronic element, and
   the first transmitted light reception signal.

What is claimed is:

1. A fluid sterilizing apparatus, comprising:
a first optoelectronic element, a first optoelectronic element for emitted light, and a first optoelectronic element for reflected light;
an optoelectronic element for transmitted light disposed to face the first optoelectronic element, the first optoelectronic element for emitted light, and the first optoelectronic element for reflected light;
a chamber including an inner space that allows passage of a fluid to be irradiated with ultraviolet light emitted from the first optoelectronic element, wherein
the chamber is disposed between the first optoelectronic element and the optoelectronic element for transmitted light,
the chamber is disposed between the first optoelectronic element for emitted light and the optoelectronic element for transmitted light, and
the chamber is disposed between the first optoelectronic element for reflected light and the optoelectronic element for transmitted light;
a first light-transmitting member, wherein
the first light-transmitting member is disposed between the first optoelectronic element and the inner space of the chamber,
the first light-transmitting member is disposed between the first optoelectronic element for emitted light and the inner space of the chamber, and
the first light-transmitting member is disposed between the first optoelectronic element for reflected light and the inner space of the chamber; and
a control unit including a processor configured to control emission of the ultraviolet light from the first optoelectronic element upon receiving
a first reflected light reception signal generated from the first optoelectronic element for reflected light upon receiving the ultraviolet light emitted from the first optoelectronic element and reflected by the first light-transmitting member,
a first emitted light reception signal generated from the first optoelectronic element for emitted light upon receiving a part of the ultraviolet light emitted from the first optoelectronic element, and
a first transmitted light reception signal generated from the optoelectronic element for transmitted light upon receiving the ultraviolet light emitted from the first optoelectronic element and subsequently transmitted through the first light-transmitting member and the fluid.

2. The fluid sterilizing apparatus according to claim 1, wherein
the first optoelectronic element is configured to emit the ultraviolet light in accordance with a drive current supplied to the first optoelectronic element, and
the processor is configured to
control a dose of ultraviolet light applied to the fluid from the first optoelectronic element to be within a predetermined range by adjusting the drive current supplied to the first optoelectronic element.

3. The fluid sterilizing apparatus according to claim 2, wherein
the control unit includes a memory storing at least a first transmitted light reception signal initial value and a predetermined transmitted light threshold, and
the processor is configured to
output a first error signal in a case that an absolute value of a difference between a magnitude of the first transmitted light reception signal and a magnitude of the first transmitted light reception signal initial value exceeds the predetermined transmitted light threshold.

4. The fluid sterilizing apparatus according to claim 3, wherein
the memory further stores a predetermined current threshold for the drive current, and
the processor is configured to
output a second error signal indicating degradation in the first optoelectronic element in a case that the absolute value of the difference between the magnitude of the first transmitted light reception signal and the magnitude of the first transmitted light reception signal initial value does not exceed the predetermined transmitted light threshold, and a magnitude of the drive current supplied to the first optoelectronic element exceeds the predetermined current threshold.

5. The fluid sterilizing apparatus according to claim 1, further comprising:
a first cover that is disposed to face the first optoelectronic element, disposed between the first optoelectronic element and the first light-transmitting member, and transmits a part of the ultraviolet light emitted from the first optoelectronic element, wherein
the first optoelectronic element for emitted light receives the ultraviolet light emitted from the first optoelectronic element and reflected by the first cover.

6. The fluid sterilizing apparatus according to claim 1, further comprising:
a first cover that is disposed to face the first optoelectronic element, disposed between the first optoelectronic element and the first light-transmitting member, and transmits a part of the ultraviolet light emitted from the first optoelectronic element, wherein
the first cover includes a first light quantity adjusting member, and the first light quantity adjusting member is disposed to face the first optoelectronic element for emitted light.

7. The fluid sterilizing apparatus according to claim 5, further comprising:
a first housing that houses the first optoelectronic element and the first optoelectronic element for emitted light, and supports the first cover.

8. The fluid sterilizing apparatus according to claim 1, wherein
the first optoelectronic element and the first optoelectronic element for reflected light are disposed to directly face the first light-transmitting member.

9. A fluid sterilizing apparatus, comprising:
one or more first optoelectronic elements, a first optoelectronic element for emitted light, and a first optoelectronic element for reflected light;
one or more second optoelectronic elements, a second optoelectronic element for emitted light, and a second optoelectronic element for reflected light that are disposed to face the one or more first optoelectronic elements, the first optoelectronic element for emitted light, and the first optoelectronic element for reflected light;
a chamber including an inner space that allows passage of a fluid to be irradiated with ultraviolet light emitted from the one or more first optoelectronic elements, wherein
the chamber is disposed between (A) and (B), wherein
(A) is a combination of the one or more first optoelectronic elements, the first optoelectronic element for emitted light, and the first optoelectronic element for reflected light, and
(B) is a combination of the one or more second optoelectronic elements, the second optoelectronic element for emitted light, and the second optoelectronic element for reflected light;
a first light-transmitting member, wherein
the first light-transmitting member is disposed between the one or more first optoelectronic elements and the inner space of the chamber,
the first light-transmitting member is disposed between the first optoelectronic element for emitted light and the inner space of the chamber, and
the first light-transmitting member is disposed between the first optoelectronic element for reflected light and the inner space of the chamber;
a second light-transmitting member, wherein
the second light-transmitting member is disposed between the one or more second optoelectronic elements and the inner space of the chamber,
the second light-transmitting member is disposed between the second optoelectronic element for emitted light and the inner space of the chamber, and
the second light-transmitting member is disposed between the second optoelectronic element for reflected light and the inner space of the chamber; and
a control unit including a processor configured to alternately switch between
a state I in which the one or more first optoelectronic elements emit the ultraviolet light and the first optoelectronic element for emitted light, the first optoelectronic element for reflected light, the one or more second optoelectronic elements, the second optoelectronic element for emitted light, and the second optoelectronic element for reflected light do not emit the ultraviolet light, and
a state II in which the one or more second optoelectronic elements emit the ultraviolet light and the one or more first optoelectronic elements, the first optoelectronic element for emitted light, the first optoelectronic element for reflected light, the second optoelectronic element for emitted light, and the second optoelectronic element for reflected light do not emit the ultraviolet light, wherein
in the state I,
the first optoelectronic element for reflected light outputs a first reflected light reception signal upon receiving the ultraviolet light emitted from the one or more first optoelectronic elements and reflected by the first light-transmitting member,
the first optoelectronic element for emitted light outputs a first emitted light reception signal upon receiving a part of the ultraviolet light emitted from the one or more first optoelectronic elements,
the second optoelectronic element for reflected light outputs a first transmitted light reception signal upon receiving the ultraviolet light emitted from the one or more first optoelectronic elements and subsequently transmitted through the first light-transmitting member, the fluid, and the second light-transmitting member, and
the processor controls emission of the ultraviolet light from the one or more first optoelectronic elements based on the first reflected light reception signal, the first emitted light reception signal, and the first transmitted light reception signal, and
in the state II,
the second optoelectronic element for reflected light outputs a second reflected light reception signal upon receiving the ultraviolet light emitted from the one or more second optoelectronic elements and reflected by the second light-transmitting member,
the second optoelectronic element for emitted light outputs a second emitted light reception signal upon receiving a part of the ultraviolet light emitted from the one or more second optoelectronic elements,
the first optoelectronic element for reflected light outputs a second transmitted light reception signal upon receiving the ultraviolet light emitted from the one or more second optoelectronic elements and subsequently transmitted through the second light-transmitting member, the fluid, and the first light-transmitting member, and
the processor controls emission of the ultraviolet light from the one or more second optoelectronic elements based on the second reflected light reception signal, the second emitted light reception signal, and the second transmitted light reception signal.

10. The fluid sterilizing apparatus according to claim 9, wherein
two or more of the first optoelectronic elements are connected in parallel, and
two or more of the second optoelectronic elements are connected in parallel.

11. The fluid sterilizing apparatus according to claim 9, wherein
the one or more first optoelectronic elements and the one or more second optoelectronic elements are configured to emit the ultraviolet light in accordance with a drive current supplied to the one or more first optoelectronic elements and the one or more second optoelectronic elements, and
the processor is configured to
control a dose of ultraviolet light applied to the fluid from the one or more first optoelectronic elements or the one or more second optoelectronic elements to be within a predetermined range by adjusting the drive current supplied to the one or more first optoelectronic elements or the one or more second optoelectronic elements.

12. The fluid sterilizing apparatus according to claim 11, wherein
the control unit includes a memory storing at least a first transmitted light reception signal initial value, a second transmitted light reception signal initial value, and a predetermined transmitted light threshold, and
the processor is configured to
output a first error signal in a case that an absolute value of a difference between a magnitude of the first transmitted light reception signal and a magnitude of the first transmitted light reception signal initial value or an absolute value of a difference between a magnitude of the second transmitted light reception signal and a magnitude of the second transmitted light reception signal initial value exceeds the predetermined transmitted light threshold.

13. The fluid sterilizing apparatus according to claim 12, wherein
the memory further stores a predetermined current threshold for the drive current, and
the processor is configured to
output a second error signal indicating degradation in the one or more first optoelectronic elements or the one or more second optoelectronic elements
in a case that the absolute value of the difference between the magnitude of the first transmitted light reception signal and the magnitude of the first transmitted light reception signal initial value does not exceed the predetermined transmitted light threshold, and a magnitude of the drive current supplied to the one or more first optoelectronic elements exceeds the predetermined current threshold; or
in a case that the absolute value of the difference between the magnitude of the second transmitted light reception signal and the magnitude of the second transmitted light reception signal initial value does not exceed the predetermined transmitted light threshold, and a magnitude of the drive current supplied to the one or more second optoelectronic elements exceeds the predetermined current threshold.

14. The fluid sterilizing apparatus according to claim 9, further comprising:
a first cover that is disposed to face the one or more first optoelectronic elements and the first optoelectronic element for emitted light, and transmits a part of the ultraviolet light emitted from the one or more first optoelectronic elements, wherein
the first cover is disposed between the one or more first optoelectronic elements and the first light-transmitting member, and
the first cover is disposed between the first optoelectronic element for emitted light and the first light-transmitting member; and
a second cover that is disposed to face the one or more second optoelectronic elements and the second optoelectronic element for emitted light, and transmits a part of the ultraviolet light emitted from the one or more second optoelectronic elements, wherein
the second cover is disposed between the one or more second optoelectronic elements and the second light-transmitting member, and
the second cover is disposed between the second optoelectronic element for emitted light and the second light-transmitting member, wherein
the first cover includes a first light quantity adjusting member, and the first light quantity adjusting member is disposed to face the first optoelectronic element for emitted light, and
the second cover includes a second light quantity adjusting member, and the second light quantity adjusting member is disposed to face the second optoelectronic element for emitted light.

15. The fluid sterilizing apparatus according to claim 14, further comprising:
a first housing that houses the one or more first optoelectronic elements and the first optoelectronic element for emitted light, and supports the first cover, and
a second housing that houses the one or more second optoelectronic elements and the second optoelectronic element for emitted light, and supports the second cover.

16. A fluid sterilizing apparatus, comprising:
a first optoelectronic element, a third optoelectronic element, a first optoelectronic element for emitted light, and a third optoelectronic element for emitted light;
a second optoelectronic element, a fourth optoelectronic element, a second optoelectronic element for emitted light, and a fourth optoelectronic element for emitted light that are disposed to face the first optoelectronic element, the third optoelectronic element, the first optoelectronic element for emitted light, and the third optoelectronic element for emitted light;
a chamber including an inner space that allows passage of a fluid to be irradiated with ultraviolet light emitted from the first optoelectronic element, wherein
the chamber is disposed between (A) and (B), wherein
(A) is a combination of the first optoelectronic element, the third optoelectronic element, the first optoelectronic element for emitted light, and the third optoelectronic element for emitted light, and
(B) is a combination of the second optoelectronic element, the fourth optoelectronic element, the second optoelectronic element for emitted light, and the fourth optoelectronic element for emitted light;
a first light-transmitting member, wherein
the first light-transmitting member is disposed between the first optoelectronic element and the inner space of the chamber,
the first light-transmitting member is disposed between the third optoelectronic element and the inner space of the chamber,
the first light-transmitting member is disposed between the first optoelectronic element for emitted light and the inner space of the chamber, and
the first light-transmitting member is disposed between the third optoelectronic element for emitted light and the inner space of the chamber;
a second light-transmitting member, wherein
the second light-transmitting member is disposed between the second optoelectronic element and the inner space of the chamber,
the second light-transmitting member is disposed between the fourth optoelectronic element and the inner space of the chamber,
the second light-transmitting member is disposed between the second optoelectronic element for emitted light and the inner space of the chamber, and
the second light-transmitting member is disposed between the fourth optoelectronic element for emitted light and the inner space of the chamber; and
a control unit including a processor configured to sequentially switch between
a first state in which the first optoelectronic element emits the ultraviolet light, and the third optoelectronic element, the first optoelectronic element for emitted light, the third optoelectronic element for emitted light, the second optoelectronic element, the fourth optoelectronic element, the second optoelectronic element for emitted light, and the fourth optoelectronic element for emitted light do not emit the ultraviolet light,
a second state in which the third optoelectronic element emits the ultraviolet light, and the first optoelectronic element, the first optoelectronic element for emitted light, the third optoelectronic element for emitted light, the second optoelectronic element, the fourth optoelectronic element, the second optoelectronic element for emitted light, and the fourth optoelectronic element for emitted light do not emit the ultraviolet light,
a third state in which the second optoelectronic element emits the ultraviolet light, and the first optoelectronic element, the third optoelectronic element, the first optoelectronic element for emitted light, the third optoelectronic element for emitted light, the fourth optoelectronic element, the second optoelectronic element for emitted light, and the fourth optoelectronic element for emitted light do not emit the ultraviolet light, and
a fourth state in which the fourth optoelectronic element emits the ultraviolet light, and the first optoelectronic element, the third optoelectronic element, the first optoelectronic element for emitted light, the third optoelectronic element for emitted light, the second optoelectronic element, the second optoelectronic element for emitted light, and the fourth optoelectronic element for emitted light do not emit the ultraviolet light, wherein
in the first state,
the third optoelectronic element outputs a third reflected light reception signal upon receiving the ultraviolet light emitted from the first optoelectronic element and reflected by the first light-transmitting member,
the first optoelectronic element for emitted light outputs a first emitted light reception signal upon receiving a part of the ultraviolet light emitted from the first optoelectronic element,
the second optoelectronic element or the fourth optoelectronic element outputs a first transmitted light reception signal upon receiving the ultraviolet light emitted from the first optoelectronic element and subsequently transmitted through the first light-transmitting member, the fluid, and the second light-transmitting member, and
the processor controls emission of the ultraviolet light from the first optoelectronic element based on the third reflected light reception signal, the first emitted light reception signal, and the first transmitted light reception signal, and
in the second state,
the first optoelectronic element outputs a first reflected light reception signal upon receiving the ultraviolet light emitted from the third optoelectronic element and reflected by the first light-transmitting member,
the third optoelectronic element for emitted light outputs a third emitted light reception signal upon receiving a part of the ultraviolet light emitted from the third optoelectronic element,
the second optoelectronic element or the fourth optoelectronic element outputs a third transmitted light reception signal upon receiving the ultraviolet light emitted from the third optoelectronic element and subsequently transmitted through the first light-transmitting member, the fluid, and the second light-transmitting member, and
the processor controls emission of the ultraviolet light from the third optoelectronic element based on the first reflected light reception signal, the third emitted light reception signal, and the third transmitted light reception signal,
in the third state,
the fourth optoelectronic element outputs a fourth reflected light reception signal upon receiving the ultraviolet light emitted from the second optoelectronic element and reflected by the second light-transmitting member,
the second optoelectronic element for emitted light outputs a second emitted light reception signal upon receiving a part of the ultraviolet light emitted from the second optoelectronic element,
the first optoelectronic element or the third optoelectronic element outputs a second transmitted light reception signal upon receiving the ultraviolet light emitted from the second optoelectronic element and subsequently transmitted through the second light-transmitting member, the fluid, and the first light-transmitting member, and
the processor controls emission of the ultraviolet light from the second optoelectronic element based on the fourth reflected light reception signal, the second emitted light reception signal, and the second transmitted light reception signal, and
in the fourth state,
the second optoelectronic element outputs a second reflected light reception signal upon receiving the ultraviolet light emitted from the fourth optoelectronic element and reflected by the second light-transmitting member,
the fourth optoelectronic element for emitted light outputs a fourth emitted light reception signal upon receiving a part of the ultraviolet light emitted from the fourth optoelectronic element,
the first optoelectronic element or the third optoelectronic element outputs a fourth transmitted light reception signal upon receiving the ultraviolet light emitted from the fourth optoelectronic element and subsequently transmitted through the second light-transmitting member, the fluid, and the first light-transmitting member, and
the processor controls emission of the ultraviolet light from the fourth optoelectronic element based on the second reflected light reception signal, the fourth emitted light reception signal, and the fourth transmitted light reception signal.

17. The fluid sterilizing apparatus according to claim 16, wherein
the first optoelectronic element, the second optoelectronic element, the third optoelectronic element, and the fourth optoelectronic element are configured to emit the ultraviolet light in accordance with a drive current supplied to the first optoelectronic element, the second optoelectronic element, the third optoelectronic element, and the fourth optoelectronic element, and
the processor is configured to
control a dose of ultraviolet light applied to the fluid from the first optoelectronic element, the second optoelectronic element, the third optoelectronic element, or the fourth optoelectronic element to be within a predetermined range by adjusting the drive current supplied to the first optoelectronic element, the second optoelectronic element, the third optoelectronic element, or the fourth optoelectronic element.

18. The fluid sterilizing apparatus according to claim 17, wherein
the control unit includes a memory storing at least a first transmitted light reception signal initial value, a third transmitted light reception signal initial value, a second transmitted light reception signal initial value, a fourth transmitted light reception signal initial value, and a predetermined transmitted light threshold, and
the processor is configured to
output a first error signal in a case that an absolute value of a difference between a magnitude of the first transmitted light reception signal and a magnitude of the first transmitted light reception signal initial value, an absolute value of a difference between a magnitude of the third transmitted light reception signal and a magnitude of the third transmitted light reception signal initial value, an absolute value of a difference between a magnitude of the second transmitted light reception signal and a magnitude of the second transmitted light reception signal initial value, or an absolute value of a difference between a magnitude of the fourth transmitted light reception signal and a magnitude of the fourth transmitted light reception signal initial value exceeds the predetermined transmitted light threshold.

19. The fluid sterilizing apparatus according to claim 18, wherein
the memory further stores a predetermined current threshold for the drive current, and
the processor is configured to
output a second error signal indicating degradation in the first optoelectronic element, the second optoelectronic element, the third optoelectronic element, or the fourth optoelectronic element
in a case that the absolute value of the difference between the magnitude of the first transmitted light reception signal and the magnitude of the first transmitted light reception signal initial value does not exceed the predetermined transmitted light threshold, and a magnitude of the drive current supplied to the first optoelectronic element exceeds the predetermined current threshold;
in a case that the absolute value of the difference between the magnitude of the third transmitted light reception signal and the magnitude of the third transmitted light reception signal initial value does not exceed the predetermined transmitted light threshold, and a magnitude of the drive current supplied to the third optoelectronic element exceeds the predetermined current threshold;
in a case that the absolute value of the difference between the magnitude of the second transmitted light reception signal and the magnitude of the second transmitted light reception signal initial value does not exceed the predetermined transmitted light threshold, and a magnitude of the drive current supplied to the second optoelectronic element exceeds the predetermined current threshold; or
in a case that the absolute value of the difference between the magnitude of the fourth transmitted light reception signal and the magnitude of the fourth transmitted light reception signal initial value does not exceed the predetermined transmitted light threshold, and a magnitude of the drive current supplied to the fourth optoelectronic element exceeds the predetermined current threshold.

20. The fluid sterilizing apparatus according to claim 16, further comprising:
a first cover that is disposed to face the first optoelectronic element, disposed between the first optoelectronic element and the first light-transmitting member, and transmits a part of the ultraviolet light emitted from the first optoelectronic element;
a second cover that is disposed to face the second optoelectronic element, disposed between the second optoelectronic element and the second light-transmitting member, and transmits a part of the ultraviolet light emitted from the second optoelectronic element;
a third cover that is disposed to face the third optoelectronic element, disposed between the third optoelectronic element and the first light-transmitting member, and transmits a part of the ultraviolet light emitted from the third optoelectronic element; and
a fourth cover that is disposed to face the fourth optoelectronic element, disposed between the fourth optoelectronic element and the second light-transmitting member, and transmits a part of the ultraviolet light emitted from the fourth optoelectronic element, wherein
the first cover includes a first light quantity adjusting member, and the first light quantity adjusting member is disposed to face the first optoelectronic element for emitted light,
the second cover includes a second light quantity adjusting member, and the second light quantity adjusting member is disposed to face the second optoelectronic element for emitted light,
the third cover includes a third light quantity adjusting member, and the third light quantity adjusting member is disposed to face the third optoelectronic element for emitted light, and
the fourth cover includes a fourth light quantity adjusting member, and the fourth light quantity adjusting member is disposed to face the fourth optoelectronic element for emitted light.

21. The fluid sterilizing apparatus according to claim 1, further comprising:
a flow meter, wherein
the processor controls the emission of the ultraviolet light from the first optoelectronic element upon further receiving a flow signal from the flow meter.
